SEQUENCE LISTING (10 sequences)

SEQ ID NO: 1 - protein, 233 aa, Staphylococcus sp
SEKSEEINEKDLRKKSELQRNALSNLRQIYYYNEKAITENKESDDQFLENTLLFKGFFTGHPWYNDLLVDLGSKDATNKYKGKKVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNKTINSENLHIDLYLYTT

SEQ ID NO: 2 - protein, 233 aa, Staphylococcus sp
SEKSEEINEKDLRKKSELQGTALGNLKQIYYYNEKAKTENKESHDQFLQHTILFKGFFTDHSWYNDLLVDFDSKDIVDKYKGKKVDLYGAYYGYQCAGGTPNKTACMYGGVTLHDNNRLTEEKKVPINLWLDGKQNTVPLETVKTNKKNVTVQELDLQARRYLQEKYNLYNSDVFDGKVQRGLIVFHTSTEPSVNYDLFGAQGQYSNTLLRIYRDNKTINSENMHIDIYLYTS

SEQ ID NO: 3 - protein, 233 aa, Artificial Sequence
  Mutated Protein
SEKSEEINEKDLRKKSELQGTALGNLKQIYYYNSKAITSSEKSADQFLTNTLLFKGFFTGHPWYNDLLVDLGSTAATSEYEGSSVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNTTISSTSLSISLYLYTT

SEQ ID NO: 4 - protein, 233 aa, Artificial Sequence
  Mutated Protein
SEKSEEINEKDLRKKSELQGTALGNLKQIYYYNEKAKTENKESHDQFLQHTILFKGFFTDHSWYNDLLVDFDSKDIVDKYKGKKVDLYGAYYGYQCAGGTPNKTACMYGGVTLHDNNRLTEEKKVPINLWLDGKQNTVPLETVKTNKKNVTVQELDLQARRYLQEKYNLYNSDVFDGKVQRGLIVFHTSTEPSVNYDLFGAQGQYSNTLLRIYRDNKTINSENMHIAIYLYTS

SEQ ID NO: 5 - protein, 679 aa, Artificial Sequence
  Conjugated Protein
  MISC_FEATURE (460)..(679): Light Chain
QVQLQQPGAELVRPGASVKLSCKASGYTFTNYWINWVKQRPGQGLEWIGNIYPSYIYTNYNQEFKDKVTLTVDESSSTAYMQLSSPTSEDSAVYYCTRSPYGYDEYGLDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSGGPSEKSEEINEKDLRKKSELQGTALGNLKQIYYYNEKAKTENKESHDQFLQHTILFKGFFTDHSWYNDLLVDFDSKDIVDKYKGKKVDLYGAYYGYQCAGGTPNKTACMYGGVTLHDNNRLTEEKKVPINLWLDGKQNTVPLETVKTNKKNVTVQELDLQARRYLQEKYNLYNSDVFDGKVQRGLIVFHTSTEPSVNYDLFGAQGQYSNTLLRIYRDNKTINSENMHIDIYLYTSDIVMTQSPSSLTVTAGEKVTMNCKSSQSLLNSRNQKNYLTWYQQKPGQPPKLLIYWASTRESGVPDRFTGSGSGTDFTLTISSVQAEDLAVYYCQNDYVYPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNES

SEQ ID NO: 6 - protein, 672 aa, Artificial Sequence
  Mutated and Conjugated Protein
  MISC_FEATURE (459)..(672): Light Chain
EVQLQQSGPDLVKPGASVKISCKASGYSFTGYYMHWVKQSHGKSLEWIGRINPNNGVTLYNQKFKDKAILTVDKSSTTAYMELRSLTSEDSAVYYCARSTMITNYVMDYWGQVTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSGGPSEKSEEINEKDLRKKSELQGTALGNLKQIYYYNEKAKTENKESHDQFLQHTILFKGFFTDHSWYNDLLVDFDSKDIVDKYKGKKVDLYGAYYGYQCAGGTPNKTACMYGGVTLHDNNRLTEEKKVPINLWLDGKQNTVPLETVKTNKKNVTVQELDLQARRYLQEKYNLYNSDVFDGKVQRGLIVFHTSTEPSVNYDLFGAQGQYSNTLLRIYRDNKTINSENMHIAIYLYTSSIVMTQTPTSLLVSAGDRVTITCKASQSVSNDVAWYQQKPGQSPKLLISYTSSRYAGVPDRFSGSGSGTDFTLTISSVQAEDLAVYFCQQDYNSPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNES

SEQ ID NO: 7 - protein, 672 aa, Artificial Sequence
  Mutated and Conjugated Protein
  MISC_FEATURE (459)..(672): Light Chain
EVQLQQSGPDLVKPGASVKISCKASGYSFTGYYMHWVKQSPGKGLEWIGRINPNNGVTLYNQKFKDKATLTVDKSSTTAYMELRSLTSEDSAVYYCARSTMITNYVMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSGGPSEKSEEINEKDLRKKSELQGTALGNLKQIYYYNSKAITSSEKSADQFLTNTLLFKGFFTGHPWYNDLLVDLGSTAATSEYEGSSVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNTTISSTSLSISLYLYTTSIVMTQTPTSLLVSAGDRVTITCKASQSVSNDVAWYQQKPGQSPKLLISYTSSRYAGVPDRFSGSGYGTDFTLTISSVQAEDAAVYFCQQDYNSPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNES

SEQ ID NO: 8 - protein, 458 aa, Artificial Sequence
  Synthetic polypeptide
EVQLQQSGPDLVKPGASVKISCKASGYSFTGYYMHWVKQSPGKGLEWIGRINPNNGVTLYNQKFKDKATLTVDKSSTTAYMELRSLTSEDSAVYYCARSTMITNYVMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSGGPSEKSEEINEKDLRKKSELQGTALGNLKQIYYYNSKAITSSEKSADQFLTNTLLFKGFFTGHPWYNDLLVDLGSTAATSEYEGSSVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNTTISSTSLSISLYLYTT

SEQ ID NO: 9 - protein, 214 aa, Artificial Sequence
  Synthetic polypeptide
SIVMTQTPTSLLVSAGDRVTITCKASQSVSNDVAWYQQKPGQSPKLLISYTSSRYAGVPDRFSGSGYGTDFTLTISSVQAEDAAVYFCQQDYNSPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNES

SEQ ID NO: 10 - protein, 233 aa, Artificial Sequence
  Mutated Protein
SEKSEEINEKDLRKKSELQGTALGNLKQIYYYNEKAITENKESDDQFLENTLLFKGFFTGHPWYNDLLVDLGSKDATNKYKGKKVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNKTINSENLHIALYLYTT

(19) United States
(12) Patent Application Publication
(10) Pub. No.: US 2024/0197784 A1
(43) Pub. Date: Jun. 20, 2024

(54) METHODS AND COMPOSITIONS FOR TREATING GLIOBLASTOMA

(71) Applicant: NeoTX Therapeutics Ltd., Rehovot (IL)

(72) Inventors: Michal Shahar, Rishon LeZion (IL); Asher Nathan, Rishon LeZion (IL)

(21) Appl. No.: 18/556,473

(22) PCT Filed: Apr. 22, 2022
(86) PCT No.: PCT/IB2022/000243

§ 371 Date: Oct. 20, 2023

Related U.S. Application Data
(60) Provisional application No. 63/178,054, filed on Apr. 22, 2021.

(51) Int. Cl. A61K 35/17; A61K 39/00; A61K 47/68; A61P 35/00; C07K 16/28; C07K 16/30

(52) U.S. Cl. CPC A61K 35/17; A61K 39/4611; A61K 39/4631; A61K 39/464402; A61K 47/6829; A61P 35/00; C07K 16/2818; C07K 16/2827; C07K 16/30; A61K 2039/507; A61K 2239/13; A61K 2239/47

(57) ABSTRACT

The disclosure provides methods and compositions for treating glioblastoma (GBM) using a 5T4-targeting agent, e.g., a superantigen conjugate comprising an anti-5T4 antibody.

### CROSS-REFERENCE TO RELATED APPLICATIONS

This application claims the benefit of and priority to U.S. Provisional Patent Application No. 63/178,054, filed Apr. 22, 2021, the disclosure of which is hereby incorporated by reference in its entirety for all purposes.

### FIELD OF THE INVENTION

The disclosure relates generally to methods and compositions for treating glioblastoma in a subject, and, more particularly, the disclosure relates to methods and compositions for treating glioblastoma using a 5T4-binding agent.

### BACKGROUND

According to the American Cancer Society, more than one million people in the United States are diagnosed with cancer each year. Cancer is a disease that results from uncontrolled proliferation of cells that were once subject to natural control mechanisms but have been transformed into cancerous cells that continue to proliferate in an uncontrolled manner.

Glioblastoma multiforme (glioblastoma or GBM) is the most common and most aggressive malignant primary brain tumor in humans. GBMs are tumors of astrocytes, which are star-shaped glial cells that form the neuronal network and perform a variety of important, active roles in the brain. GBMs are highly lethal and characterized by extensive necrosis as well as a high rate of angiogenesis. Patients diagnosed with GBM have poor prognosis and survival rate. Treatment typically involves resection, chemotherapy, or radiation. Median survival with standard of care (Temozolomide) is 15 months, with a two-year survival of less than 25%. Survival without treatment is usually only a few months. Furthermore, the Human Protein Atlas states that malignant gliomas exhibit no membranous or cytoplasmic immunoreactivity for 5T4 (TPBG) expression (see, world wide web proteinatlas.org/ENSG00000146242-TPBG/pathology).

Despite the significant advances being made in cancer treatment and management, there is still an ongoing need for new and effective therapies for treating and managing cancers, e.g., glioblastomas.

### SUMMARY OF THE INVENTION

The invention is based, in part, upon the discovery that glioblastoma (GBM) tumor samples and cell lines exhibit significant levels of 5T4 expression, and that an anti-cancer treatment including a 5T4-targeting agent (for example, a superantigen conjugate including an anti-5T4 antibody) can be used to treat GBM.

Accordingly, the invention provides a method of (i) reducing tumor volume in a subject with glioblastoma, (ii) killing tumor cells in a subject with glioblastoma, and/or (iii) treating glioblastoma in a subject in need of treatment. The foregoing methods include administering to the subject an effective amount of a 5T4-targeting agent.

In certain embodiments of any of the foregoing methods, at least 0.5%, 1%, 2%, 3%, 4%, 5%, 10%, 15%, 20%, 25% or 30% of (i) tumor cells in the subject, (ii) tumor cells in a tumor in the subject, and/or (iii) tumor cells in a tumor sample (e.g., a tumor tissue sample) from the subject, exhibit cell membrane expression of 5T4, as measured by immunohistochemistry. The existence of membrane expression versus cytoplasmic expression of 5T4 is significant when 5T4 is used a target for targeted therapy of cancerous cells (e.g., GBM cells).

In certain embodiments of any of the foregoing methods, the 5T4-targeting agent is or comprises an antibody, a bispecific T-cell engager (BiTE), an immune cell, or a vaccine. In certain embodiments, the 5T4-targeting agent is or comprises a superantigen conjugate comprising a superantigen covalently linked to an anti-5T4 antibody. The superantigen can comprise Staphylococcal enterotoxin A or an immunologically reactive variant and/or fragment thereof. In certain embodiments, the superantigen comprises the amino acid sequence of SEQ ID NO: 3, or an immunologically reactive variant and/or fragment thereof. In certain embodiments, the anti-5T4 antibody comprises a heavy chain comprising amino acid residues 1-458 of SEQ ID NO: 8 and a light chain comprising amino acid residues 1-214 of SEQ ID NO: 9. In certain embodiments, the superantigen conjugate comprises a first protein chain comprising SEQ ID NO: 8 and a second protein chain comprising SEQ ID NO: 9.

In certain embodiments of any of the foregoing methods, the 5T4-targeting agent is or comprises an immune cell (e.g., an isolated immune cell) that can be, for example, a T-cell, a natural killer cell (NK), or a natural killer T-cell (NKT). In certain embodiments, the immune cell is a chimeric antigen receptor (CAR)-expressing immune cell.

In certain embodiments of any of the foregoing methods, the method further comprises administering to the subject an immunopotentiator, which can be, for example, a CTLA-4-based inhibitor or a PD-1-based inhibitor. In certain embodiments, the PD-1 based inhibitor is a PD-1 or PD-L1 inhibitor. In certain embodiments, the PD-1 inhibitor is an anti-PD-1 antibody, e.g., an anti-PD-1 antibody selected from nivolumab pembrolizumab, and cemiplimab. In certain embodiments, the PD-L1 inhibitor is an anti-PD-L1 antibody, e.g., an anti-PD-L1 antibody selected from atezolizumab, avelumab, and durvalumab.

These and other aspects and features of the invention are described in the following detailed description and claims.

### DESCRIPTION OF THE DRAWINGS

The invention can be more completely understood with reference to the following drawings.

FIG. 1 is a sequence alignment showing the homologous A-E regions in certain wild type and modified superantigens.

FIG. 2 is a schematic depiction of an exemplary superantigen conjugate, naptumomab estafenatox/ANYARA®.

FIG. 3 is an amino acid sequence corresponding to an exemplary superantigen conjugate, naptumomab estafenatox/ANYARA®, which comprises two protein chains. The first protein chain comprises residues 1 to 458 of SEQ ID NO: 7 (see also, SEQ ID NO: 8), and includes a chimeric 5T4 Fab heavy chain, corresponding to residues 1 to 222 of SEQ ID NO: 7, and the SEA/E-120 superantigen, corresponding to residues 226 to 458 of SEQ ID NO: 7, covalently linked via a GGP tripeptide linker, corresponding to residues 223-225 of SEQ ID NO: 7. The second chain comprises residues 459 to 672 of SEQ ID NO: 7 (see also, SEQ ID NO: 9) and includes a chimeric 5T4 Fab light chain. The two protein chains are held together by non-covalent interactions between the Fab heavy and light chains.

FIG. 4A-4B are representative immunohistochemistry (IHC) images (A-0.5X, B-20X) of a glioblastoma tumor section stained with an antibody against either 5T4 (left) or a control IgG antibody (right). In this tumor section, 20% of the glioblastoma cells exhibited membranal 5T4 expression. Bars at the top left of each image indicate image scale (A 2000 μm; B 100 μm). The box in FIG. 4A indicates the region in the tumor section that was magnified and shown in FIG. 4B.

FIG. 5 depicts 5T4 expression on U-138 (GBM), U-87 (GBM) GMS-10 (GBM), Caki-2 (RCC: high 5T4-expression control), and OVCAR-3 (Ovarian cancer, low 5T4-expression control) cell lines as measured by flow cytometry. FC indicates fold change of 5T4 antibody staining over isotype control antibody staining.

FIGS. 6A-C are a series of dose-response curves illustrating the anti-cancer effect of naptumomab estafenatox (NAP) in combination with T cells against glioblastoma (GBM) cell lines. GBM cells were treated with NAP (0-10 μg/ml) for 48 hours and viable cells were counted. Dose-response curves are shown for three GBM cell lines: U-138 (FIG. 6A), U-87 (FIG. 6B), and GMS-10 (FIG. 6C). The IC50 (the NAP concentration that inhibits 50% of cancer cell viability) was found to be 0.5 ng/ml, 0.3 ng/ml, and 0.95 ng/ml for U-138, U-87, and GMS-10, respectively.

FIG. 7 is a series of viability curves demonstrating the effect of an anti-5T4 antibody on the anti-cancer effect of naptumomab estafenatox (NAP) against the glioblastoma GMS-10 cell line. Fluorescently-stained GMS-10 cells were treated with an anti 5T4 Fab (0-50 μg/ml) for 24 hours to block the 5T4 antigen. GMS-10 cells were subsequently treated with NAP (0-0.005 μg/ml) and T cells were added at an effector:target (T cell:GMS-10 cell) ratio of 10:1 for 48 hours. Viability of the cancer cells (%) was determined by FACS. Viability of the cancer cells (%)=(cell count value of treatment group/cell count value of control group)×100.

### DETAILED DESCRIPTION

The invention is based, in part, upon the discovery that glioblastoma (GBM) tumor samples and cell lines exhibit significant levels of 5T4 expression (in particular membrane expression of 5T4), and that an anti-cancer treatment using a 5T4-targeting agent (for example, a superantigen conjugate including an anti-5T4 antibody) can be used to treat GBM. This discovery was unexpected because the membranous and cytoplasmic immunoreactivity of 5T4 expression in malignant gliomas was considered to be negative (see, Human Protein Atlas, supra). As described in Example 1, it has been surprisingly discovered that a predominant number of GBM tissue samples evaluated (greater than 80%) exhibited membrane 5T4 expression positivity. Furthermore, it has also been discovered that the 5T4 antigen degrades quickly in cellular and/or tissue samples. Without wishing to be bound by theory, it is possible that prior studies may not have identified the significance of membrane-bound 5T4 given its instability ex vivo.

Accordingly, in one aspect, the invention provides a method of reducing tumor volume in a subject with glioblastoma. In another aspect, the invention provides a method of killing tumor cells in a subject with glioblastoma. In another aspect, the invention provides a method of treating glioblastoma in a subject in need thereof. In each of the foregoing aspects, the method comprises administering to the subject an effective amount of a 5T4-targeting agent.

Also provided is a method of identifying a subject suitable for treatment with a 5T4 targeting agent. The subject can be identified based on the expression (and expression level) of membranous 5T4 in cell sample and/or tissue sample harvested from a subject. The expression and expression levels of membranous 5T4 can be determined by various techniques in the art, e.g., immunohistochemical studies. Depending upon the circumstances, subjects with detectable levels of membranous 5T4 are suitable for treatment with a 5T4 targeting agent.

In certain circumstances, at least 0.5%, 1%, 2%, 3%, 4%, 5%, 10%, 15%, 20%, 25%, 30%, 35%, 40%, 45%, 50%, 55%, 60%, 65%, 70%, 75%, 80%, 85% or 90% of (i) tumor cells in the subject, (ii) tumor cells in a tumor in the subject, and/or (iii) tumor cells in a tumor sample (e.g., a tumor tissue sample) from the subject, exhibit cell membrane expression of 5T4, as measured by immunohistochemistry and/or fluorescence-activated cell sorting (FACS).

In certain embodiments, from about 0.5% to about 80%, from about 0.5% to about 60%, from about 0.5% to about 40%, from about 0.5% to about 20%, from about 0.5% to about 15%, from about 0.5% to about 10%, from about 0.5% to about 5%, from about 0.5% to about 4%, from about 0.5% to about 3%, from about 0.5% to about 2%, from about 0.5% to about 1%, from about 1% to about 80%, from about 1% to about 60%, from about 1% to about 40%, from about 1% to about 20%, from about 1% to about 15%, from about 1% to about 10%, from about 1% to about 5%, from about 1% to about 4%, from about 1% to about 3%, from about 1% to about 2%, from about 2% to about 80%, from about 2% to about 60%, from about 2% to about 40%, from about 2% to about 20%, from about 2% to about 15%, from about 2% to about 10%, from about 2% to about 5%, from about 2% to about 4%, from about 2% to about 3%, from about 3% to about 80%, from about 3% to about 60%, from about 3% to about 40%, from about 3% to about 20%, from about 3% to about 15%, from about 3% to about 10%, from about 3% to about 5%, from about 3% to about 4%, from about 4% to about 80%, from about 4% to about 60%, from about 4% to about 40%, from about 4% to about 20%, from about 4% to about 15%, from about 4% to about 10%, from about 4% to about 5%, from about 5% to about 80%, from about 5% to about 60%, from about 5% to about 40%, from about 5% to about 20%, from about 5% to about 15%, from about 5% to about 10%, from about 10% to about 80%, from about 10% to about 60%, from about 10% to about 40%, from about 10% to about 20%, from about 10% to about 15%, from about 15% to about 80%, from about 15% to about 60%, from about 15% to about 40%, from about 15% to about 20%, from about 20% to about 80%, from about 20% to about 60%, from about 20% to about 40%, from about 40% to about 80%, from about 40% to about 60%, or from about 60% to about 80%, of (i) tumor cells in the subject, (ii) tumor cells in a tumor in the subject, and/or (iii) tumor cells in a tumor sample (e.g., a tumor tissue sample) from the subject, exhibit cell membrane expression of 5T4, as measured by immunohistochemistry and/or fluorescence-activated cell sorting (FACS).

The level of 5T4 membrane expression exhibited by the tumor cells can be measured, e.g., by flow cytometry, e.g., as described in Example 2 herein. In certain embodiments. 5T4 membrane expression is quantified as the fold-increase (also referred to as fold change or FC) in binding of an anti-5T4 antibody to a tumor cell relative to an isotype control. For example, in certain embodiments, there is about a 1.5-fold increase, about a 2-fold increase, about a 2.5-fold increase, about a 3-fold increase, about a 4-fold increase, about a 5-fold increase, about a 6-fold increase, about a 7-fold increase, about an 8-fold increase, about a 9-fold increase, about a 10-fold increase, about an 11-fold increase, about a 12-fold increase, about a 13-fold increase, about a 14-fold increase, about a 15-fold increase, about a 16-fold increase, about a 17-fold increase, about an 18-fold increase, about a 19-fold increase, about a 20-fold increase, or greater than about a 20-fold increase in binding of anti-5T4 antibody to the tumor cell relative to an isotype control, as measured by, e.g., flow cytometry. In certain embodiments, (i) tumor cells in the subject, (ii) tumor cells in a tumor in the subject, and/or (iii) tumor cells in a tumor sample (e.g., a tumor tissue sample) from the subject exhibit about the same or a greater level of cell membrane expression of 5T4 than a low-5T4-expression control, e.g., OVCAR-3, but about the same as or a lower level of cell membrane expression of 5T4 than a high-5T4-expression control, e.g., Caki-2.

In certain embodiments, (i) tumor cells in the subject, (ii) tumor cells in a tumor in the subject, and/or (iii) tumor cells in a tumor sample (e.g., a tumor tissue sample) from the subject exhibit low membrane expression of 5T4, e.g., as measured by flow cytometry. For example, in certain embodiments, the tumor cells exhibit lower cell membrane expression of 5T4 or about the same level of cell membrane expression of 5T4 as an appropriate low-5T4-expression control, e.g., OVCAR-3, and/or lower cell membrane expression of 5T4 than an appropriate high-5T4-expression control, e.g., Caki-2.

In certain embodiments, (i) tumor cells in the subject, (ii) tumor cells in a tumor in the subject, and/or (iii) tumor cells in a tumor sample (e.g., a tumor tissue sample) from the subject exhibit medium membrane expression of 5T4, e.g., as measured by flow cytometry. For example, in certain embodiments, the tumor cells exhibit higher cell membrane expression of 5T4 than an appropriate low-5T4-expression control, e.g., OVCAR-3, and/or lower cell membrane expression of 5T4 than an appropriate high-5T4-expression control, e.g., Caki-2.

In certain embodiments, (i) tumor cells in the subject, (ii) tumor cells in a tumor in the subject, and/or (iii) tumor cells in a tumor sample (e.g., a tumor tissue sample) from the subject exhibit high membrane expression of 5T4, e.g., as measured by flow cytometry. For example, in certain embodiments, the tumor cells exhibit higher cell membrane expression of 5T4 or about the same level of cell membrane expression of 5T4 as an appropriate high-5T4-expression control, e.g., Caki-2, and/or higher cell membrane expression of 5T4 than an appropriate low-5T4-expression control, e.g., OVCAR-3.

In certain embodiments, (i) tumor cells in the subject, (ii) tumor cells in a tumor in the subject, and/or (iii) tumor cells in a tumor sample (e.g., a tumor tissue sample) from the subject exhibit a lower level, about the same level, or a higher level of 5T4 membrane expression than a glioblastoma cell line, e.g., as measured by flow cytometry. In certain embodiments, the glioblastoma cell line is U-138, U87, or GMS-10.

In certain embodiments of any of the foregoing methods, the 5T4-targeting agent is or comprises an antibody, a bispecific T-cell engager (BiTE), an immune cell (e.g., a T cell, e.g., a CAR-T cell), or a vaccine.

In certain embodiments, the 5T4-targeting agent does not remove or otherwise inhibit cancer stem cells. In certain embodiments, the 5T4-targeting agent does not primarily mediate treatment of the glioblastoma in the subject by removing or otherwise inhibiting cancer stem cells.

Various features and aspects of the invention are discussed in more detail below.

### I. Definitions

Unless defined otherwise, technical and scientific terms used herein have the same meaning as commonly understood by one of ordinary skill in the art to which this invention belongs. For purposes of the present invention, the following terms are defined below.

As used herein, the terms “a” or “an” may mean one or more. For example, a statement such as “treatment with a superantigen and an immune cell,” can mean treatment, with one superantigen and immune cell; with more than one superantigen and one immune cell; with one superantigen and more than one immune cell; or with more than one superantigen and more than one immune cell.

As used herein, unless otherwise indicated, the term “antibody” is understood to mean an intact antibody (e.g., an intact monoclonal antibody) or antigen-binding fragment of an antibody, including an intact antibody or antigen-binding fragment of an antibody (e.g., a phage display antibody including a fully human antibody, a semisynthetic antibody or a fully synthetic antibody) that has been optimized, engineered or chemically conjugated. Examples of antibodies that have been optimized are affinity-matured antibodies. Examples of antibodies that have been engineered are Fc optimized antibodies, antibodies engineered to reduce immunogenicity, and multi-specific antibodies (e.g., bispecific antibodies). Examples of antigen-binding fragments include Fab, Fab′, F(ab′)₂, Fv, single chain antibodies (e.g., scFv), minibodies and diabodies. An antibody conjugated to a toxin moiety is an example of a chemically conjugated antibody.

As used herein, the terms “cancer” and “cancerous” are understood to mean the physiological condition in mammals that is typically characterized by unregulated cell growth. Examples of cancer include, but are not limited to, melanoma, carcinoma, lymphoma, blastoma, sarcoma, and leukemia or lymphoid malignancies. More particular examples of cancers include squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, bone cancer, brain cancer, retinoblastoma, endometrial cancer or uterine carcinoma, salivary gland carcinoma, kidney or renal cancer, prostate cancer, vulval cancer, thyroid cancer, hepatic carcinoma, anal carcinoma, penile carcinoma, testicular cancer, as well as head and neck cancer, gum or tongue cancer. The cancer comprises cancer or cancerous cells, for example, the cancer may comprise a plurality of individual cancer or cancerous cells, for example, a leukemia, or a tumor comprising a plurality of associated cancer or cancerous cells.

As used herein, the terms “cancer stem cell” or “CSC” refer to a sub-population of cancer cells that possesses characteristics normally associated with stem cells, such as self-renewal, the ability to differentiate into multiple cell types and give rise to multiple cancer cell types, indefinite life span and abbreviated cell cycle regulation. CSCs are tumorigenic and are capable of forming tumors from small number of cells in animal tumor models. CSCs can persist in tumors as a distinct sub-population and cause relapse and metastasis by giving rise to new tumors.

As used herein, the term “refractory” refers to a cancer that does not respond or no longer responds to a treatment. In certain embodiments, a refractory cancer can be resistant to a treatment before or at the beginning of the treatment. In other embodiments, the refractory cancer can become resistant during or after a treatment. A refractory cancer is also called a resistant cancer. As used herein, the term “recurrence” or “relapse” refers to the return of a refractory cancer or the signs and symptoms of a refractory cancer after a positive response a prior treatment (e.g., a reduction in tumor burden, a reduction in tumor volume, a reduction in tumor metastasis, or a modulation of a biomarker indicative of a positive response to a treatment).

As used herein, the term “immunogen” is a molecule that provokes (evokes, induces, or causes) an immune response. This immune response may involve antibody production, the activation of certain cells, such as, for example, specific immunologically-competent cells, or both. An immunogen may be derived from many types of substances, such as, but not limited to, molecules from organisms, such as, for example, proteins, subunits of proteins, killed or inactivated whole cells or lysates, synthetic molecules, and a wide variety of other agents both biological and nonbiological. It is understood that essentially any macromolecule (including naturally occurring macromolecules or macromolecules produced via recombinant DNA approaches), including virtually all proteins, can serve as immunogens.

As used herein, the term “immunogenicity” relates to the ability of an immunogen to provoke (evoke, induce, or cause) an immune response. Different molecules may have differing degrees of immunogenicity, and a molecule having an immunogenicity that is greater compared to another molecule is known, for example, to be capable of provoking (evoking, inducing, or causing) a greater immune response than would an agent having a lower immunogenicity.

As used herein, the term “antigen” as used herein refers to a molecule that is recognized by antibodies, specific immunologically-competent cells, or both. An antigen may be derived from many types of substances, such as, but not limited to, molecules from organisms, such as, for example, proteins, subunits of proteins, nucleic acids, lipids, killed or inactivated whole cells or lysates, synthetic molecules, and a wide variety of other agents both biological and non-biological.

As used herein, the term “antigenicity” relates to the ability of an antigen to be recognized by antibodies, specific immunologically-competent cells, or both.

As used herein, the term “epitope spreading” refers to the diversification of the epitope specificity of an immune response from an initial epitope-specific immune response directed against an antigen to other epitopes on that antigen (intramolecular spreading) or other antigens (intermolecular spreading). Epitope spreading allows a subject's immune system to determine additional target epitopes not initially recognized by the immune system in response to the original therapeutic protocol while reducing the possibility of escape variants in a tumor population and thus affect progression of disease.

As used herein, the term “immune response” refers to a response by a cell of the immune system, such as a B cell, T cell (CD4+ or CD8+), regulatory T cell, antigen-presenting cell, dendritic cell, monocyte, macrophage. NKT cell, NK cell, basophil, eosinophil, or neutrophil, to a stimulus. In some embodiments, the response is specific for a particular antigen (an “antigen-specific response”), and refers to a response by a CD4+ T cell, CD8+ T cell, or B cell via their antigen-specific receptor. In some embodiments, an immune response is a T cell response, such as a CD4+ response or a CD8+ response. Such responses by these cells can include, for example, cytotoxicity, proliferation, cytokine or chemokine production, trafficking, or phagocytosis, and can be dependent on the nature of the immune cell undergoing the response.

As used herein, the term “major histocompatibility complex,” or “MHC,” refers to a specific cluster of genes, many of which encode evolutionarily related cell surface proteins involved in antigen presentation, that are important determinants of histocompatibility. Class I MHC, or MHC-I, function mainly in antigen presentation to CD8⁺ T lymphocytes (CD8′ T-Cells). Class II MHC, or MHC-II, function mainly in antigen presentation to CD4⁺ T lymphocytes (CD4⁺ T-Cells).

As used herein, the term “derived,” for example “derived from,” includes, but is not limited to, for example, wild-type molecules derived from biological hosts such as bacteria, viruses and eukaryotic cells and organisms, and modified molecules, for example, modified by chemical means or produced in recombinant expression systems.

As used herein, the terms “seroreactive,” “seroreaction” or “seroreactivity” are understood to mean the ability of an agent, such as a molecule, to react with antibodies in the serum of a mammal, such as, but not limited to, a human. This includes reactions with all types of antibodies, including, for example, antibodies specific for the molecule and nonspecific antibodies that bind to the molecule, regardless of whether the antibodies inactivate or neutralize the agent. As is known in the art, different agents may have different seroreactivity relative to one another, wherein an agent having a seroreactivity lower than another would, for example, react with fewer antibodies and/or have a lower affinity and/or avidity to antibodies than would an agent having a higher seroreactivity. This may also include the ability of the agent to elicit an antibody immune response in an animal, such as a mammal, such as a human.

As used herein, the terms “soluble T-cell receptor,” or “soluble TCR,” are understood to mean a “soluble” T-cell receptor comprising the chains of a full-length (e.g., membrane bound) receptor, except that the transmembrane region of the receptor chains are deleted or mutated so that the receptor, when expressed by a cell, will not insert into, traverse or otherwise associate with the membrane. A soluble T-cell receptor may comprise only the extracellular domains or extracellular fragments of the domains of the wild-type receptor (e.g., lacks the transmembrane and cytoplasmic domains).

As used herein, the term “superantigen” is understood to mean a class of molecules that stimulate a subset of T-cells by binding to MHC class II molecules and VD domains of T-cell receptors, thereby activating T-cells expressing particular VO gene segments. The term includes wild-type, naturally occurring superantigens, for example, those isolated from certain bacteria or expressed from unmodified genes from same, as well as modified superantigens, wherein, for example, the DNA sequence encoding a superantigen has been modified, for example, by genetic engineering, to, for example, produce a fusion protein with a targeting moiety, and/or alter certain properties of the superantigen, such as, but not limited to, its MHC class II binding (for example, to reduce affinity) and/or its seroreactivity, and/or its immunogenicity, and/or antigenicity (for example, to reduce its seroreactivity). The definition includes wild-type and modified superantigens and any immunologically reactive variants and/or fragments thereof described herein or in the following U.S. patents and patent applications: U.S. Pat. Nos. 5,858,363, 6,197,299, 6,514,498, 6,713,284, 6,692,746, 6,632,640, 6,632,441, 6,447,777, 6,399,332, 6,340,461, 6,338,845, 6,251,385, 6,221,351, 6,180,097, 6,126,945, 6,042,837, 6,713,284, 6,632,640, 6,632,441, 5,859,207, 5,728,388, 5,545,716, 5,519,114, 6,926,694, 7,125,554, 7,226,595, 7,226,601, 7,094,603, 7,087,235, 6,835,818, 7,198,398, 6,774,218, 6,913,755, 6,969,616, and 6,713,284, U.S. Patent Application Nos. 2003/0157113, 2003/0124142, 2002/0177551, 2002/0141981, 2002/0115190, 2002/0051765, and 2001/0046501, and PCT International Publication Number WO/03/094846.

As used herein, the term “targeting moiety” refers to any structure, molecule or moiety that is able to bind to a cellular molecule, for example, a cell surface molecule, preferably a disease specific molecule such as an antigen expressed preferentially on a cancer (or cancerous) cell. Exemplary targeting moieties include, but are not limited to, antibodies (including antigen binding fragments thereof) and the like, soluble T-cell receptors, interleukins, hormones, and growth factors.

As used herein, the terms “tumor-targeted superantigen” or “TTS” or “cancer-targeted superantigen” are understood to mean a molecule comprising one or more superantigens covalently linked (either directly or indirectly) with one or more targeting moieties.

As used herein, the term “T-cell receptor” is understood to mean a receptor that is specific to T-cells, and includes the understanding of the term as known in the art. The term also includes, for example, a receptor that comprises a disulfide-linked heterodimer of the highly variable α or β chains expressed at the cell membrane as a complex with the invariant CD3 chains, and a receptor made up of variable γ and δ chains expressed at the cell membrane as a complex with CD3 on a subset of T-cells.

As used herein, the terms “therapeutically effective amount” and “effective amount,” are understood to mean an amount of an active agent, for example, a pharmaceutically active agent or a pharmaceutical composition that produces at least some effect in treating a disease or a condition. The effective amount of pharmaceutically active agent(s) used to practice the present invention for a therapeutic treatment varies depending upon the manner of administration, the age, body weight, and general health of the subject. An effective amount can be administered in one or more administrations, applications or dosages and is not intended to be limited to a particular formulation or administration route.

As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably includes humans.

As used herein, the terms “treat,” “treating” and “treatment” are understood to mean the treatment of a disease in a mammal, e.g., in a human. This includes: (a) inhibiting the disease, i.e., arresting its development; and (b) relieving the disease, i.e., causing regression of the disease state; and (c) curing the disease. As used in the context of a therapeutic treatment, the terms “prevent” or “block” are understood to completely prevent or block, or not completely prevent or block (e.g., partially prevent or block) a given act, action, activity, or event.

As used herein, the term “inhibits the growth of a cancer” is understood to mean a measurably slowing, stopping, or reversing the growth rate of the cancer or cancerous cells in vitro or in vivo. Desirably, the growth rate is slowed by 20%, 30%, 50%, or 70% or more, as determined using a suitable assay for determination of cell growth rates. Typically, a reversal of growth rate is accomplished by initiating or accelerating necrotic or apoptotic mechanisms of cell death in neoplastic cells, resulting in a shrinkage of a neoplasm.

As used herein, the terms “variant,” “variants,” “modified,” “altered,” “mutated,” and the like, are understood to mean proteins or peptides and/or other agents and/or compounds that differ from a reference protein, peptide or other compound. Variants in this sense are described below and elsewhere in greater detail. For example, changes in a nucleic acid sequence of the variant may be silent, e.g., they may not alter the amino acids encoded by the nucleic acid sequence. Where alterations are limited to silent changes of this type a variant will encode a peptide with the same amino acid sequence as the reference peptide. Changes in the nucleic acid sequence of the variant may alter the amino acid sequence of a peptide encoded by the reference nucleic acid sequence. Such nucleic acid changes may result in amino acid substitutions, additions, deletions, fusions and/or truncations in the protein or peptide encoded by the reference sequence, as discussed below. Generally, differences in amino acid sequences are limited so that the sequences of the reference and the variant are similar overall and, in many regions, identical. A variant and reference protein or peptide may differ in amino acid sequence by one or more substitutions, additions, deletions, fusions and/or truncations, which may be present in any combination. A variant may also be a fragment of a protein or peptide of the invention that differs from a reference protein or peptide sequence by being shorter than the reference sequence, such as by a terminal or internal deletion. Another variant of a protein or peptide of the invention also includes a protein or peptide which retains essentially the same function or activity as the reference protein or peptide. A variant may also be: (i) one in which one or more of the amino acid residues are substituted with a conserved or non-conserved amino acid residue and such substituted amino acid residue may or may not be one encoded by the genetic code, or (ii) one in which one or more of the amino acid residues includes a substituent group, or (iii) one in which the mature protein or peptide is fused with another compound, such as a compound to increase the half-life of the protein or peptide (for example, polyethylene glycol), or (iv) one in which the additional amino acids are fused to the mature protein or peptide, such as a leader or secretory sequence or a sequence which is employed for purification of the mature protein or peptide. Variants may be made by mutagenesis techniques, and/or altering mechanisms such as chemical alterations, fusions, adjuncts and the like, including those applied to nucleic acids, amino acids, cells or organisms, and/or may be made by recombinant means.

As used herein, the term “sequential dosage” and related terminology refers to the administration of at least one agent (e.g., a superantigen conjugate), with at least one additional agent (e.g., an immune cell), and includes staggered doses of these agents (i.e., time-staggered) and variations in dosage amounts. This includes one agent being administered before, overlapping with (partially or totally), or after administration of another agent. In addition, the term “sequential dosage” and related terminology also includes the administration of at least one superantigen, one immune cell and more or more optional additional compounds such as, for example, a corticosteroid, an immune modulator, and another agent designed to reduce potential immunoreactivity to the superantigen conjugate administered to the subject.

As used herein, the terms “systemic” and “systemically” in the context of administration are understood to mean administration of an agent such that the agent is exposed to at least one system associated with the whole body, such as but not limited to the circulatory system, immune system, and lymphatic system, rather than only to a localized part of the body, such as but not limited to within a tumor. Thus, for example, a systemic therapy or an agent administered systematically is a therapy or an agent in which at least one system associated with the entire body is exposed to the therapy or agent, as opposed to, rather than just a target tissue.

As used herein, the term “parenteral administration” includes any form of administration in which the compound is absorbed into the subject without involving absorption via the intestines. Exemplary parenteral administrations that are used in the present invention include, but are not limited to intramuscular, intravenous, intraperitoneal, or intraarticular administration.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

At various places in the present specification, values are disclosed in groups or in ranges. It is specifically intended that the description include each and every individual subcombination of the members of such groups and ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

### II. 5T4-Targeting Agents

As discussed above, the invention is based, in part, upon the discovery that glioblastoma tissue can express significant levels of 5T4 (in particular, significant levels of membranous 5T4). As a result, 5T4-targeting agents can be used in targeting glioblastoma cells for therapy.

5T4 is a 72 kDa oncofetal glycoprotein that is expressed on certain cancers. For example, immunohistochemical analysis indicates that 5T4 expression is an indicator of poor prognosis in colorectal cancer, ovarian cancer, gastric cancer and non-small cell lung cancer. The nucleotide and amino acid sequences of human 5T4 are available at GenBank at accession no. Z29083. Sequence analysis has identified 5T4 as a member of the leucine rich repeat (LRR) family of proteins. The protein contains a short cytoplasmic tail of 44 amino acids and an extracellular domain including two leucine rich repeat (LRR) regions with associated cysteine containing flanking regions. All of the seven consensus N×S/T N-glycosylation sites in the extracellular domain are glycosylated with a combination of complex glycans, including two high mannose chains and five sialylated, bi- to tetra-antennary complex chains with minor quantities of core fucosylation.

The term “5T4-targeting agent” as used herein, refers to an agent that directs a therapy to the 5T4 antigen. The targeting agent may, for example, bind to 5T4, or alternatively may, for example, mediate the generation of additional molecules that bind to 5T4. For example, the term 5T4-targeting agent encompasses a vaccine that mediates the generation of an immune response in a subject against the 5T4 antigen.

In certain embodiments, the targeting agent binds (e.g., directly binds) to 5T4. In such embodiments, the targeting agent may alternatively be referred to as a binding agent. The term “binding agent” as used herein refers to an agent that binds preferentially or specifically to an analyte of interest (e.g., 5T4). The terms “bind preferentially,” or “binds specifically” as used in connection with a binding agent refers to an agent that binds and/or associates (i) more stably, (ii) more rapidly, (iii) with stronger affinity, (iv) with greater duration, or (v) or a combination of any two or more of (i)-(iv), with a particular target analyte it does with a molecule other than the target analyte. For example, a binding agent that specifically or preferentially binds a target analyte is a binding domain that binds a target analyte, e.g., with stronger affinity, avidity, more readily, and/or with greater duration than it binds a different analyte. The binding agent may have an affinity for the analyte of about 100 nM, 50 nM, 20 nM, 15 nM, 10 nM, 9 nM, 8 nM, 7 nM, 6 nM, 5 nM, 4 nM, 3 nM, 2 nM, 1 nM, 0.5 nM, 0.1 nM, or 0.01 nM, or stronger, as determined by surface plasmon resonance. For example, the binding agent may have an affinity for the analyte within the range from about 0.01 nM to about 100 nM, from about 0.1 nM to about 100 nM, or from about 1 nM to about 100 nM. It is understood that a binding agent that binds preferentially to a first target analyte may or may not preferentially bind to a second target analyte. As such, “preferential binding” does not necessarily require (although it can include) exclusive binding.

Exemplary binding agents include enzymes (for example, that bind substrates and inhibitors), antibodies (for example, that bind antigens), antigens (for example, that bind target antibodies), receptors (for example, that bind ligands), ligands (for example, that bind receptors), nucleic acid single-strand polymers (for example, that bind nucleic acid molecules to form, for example, DNA-DNA. RNA-RNA, or DNA-RNA double strands), and synthetic molecules that bind with target analytes. Natural, synthetic, semi-synthetic, and genetically-altered macromolecules may be employed as binding agents. Binding agents include biological binding agents, for example, an antibody, an aptamer, a receptor, an enzyme, or a nucleic acid.

### A. Antibodies

In general, antibodies are multimeric proteins that contain four polypeptide chains. Two of the polypeptide chains are called immunoglobulin heavy chains (H chains), and two of the polypeptide chains are called immunoglobulin light chains (L chains). The immunoglobulin heavy and light chains are connected by an interchain disulfide bond. The immunoglobulin heavy chains are connected by interchain disulfide bonds. A light chain consists of one variable region (V_{L}) and one constant region (C_{L}). The heavy chain consists of one variable region (V_{H}) and at least three constant regions (CH₁, CH₂ and CH₃). The variable regions determine the binding specificity of the antibody.

Each variable region contains three hypervariable regions known as complementarity determining regions (CDRs) flanked by four relatively conserved regions known as framework regions (FRs). The extent of the FRs and CDRs has been defined (Kabat, E. A., et al. (1991) SEQUENCES OF PROTEINS OF IMMUNOLOGICAL INTEREST, FIFTH EDITION. U.S. Department of Health and Human Services, NIH Publication No. 91-3242; and Chothia, C. et al. (1987) J. MOL. BIOL. 196:901-917). The three CDRs, referred to as CDR₁, CDR₂, and CDR₃, contribute to the antibody binding specificity. Naturally occurring antibodies have been used as starting material for engineered antibodies, such as chimeric antibodies and humanized antibodies.

As used herein, an antibody can include an intact antibody (e.g., an intact monoclonal antibody), or a fragment thereof, such as a Fc fragment of an antibody (e.g., an Fc fragment of a monoclonal antibody), or an antigen-binding fragment of an antibody (e.g., an antigen-binding fragment of a monoclonal antibody), including an intact antibody, antigen-binding fragment, or Fc fragment that has been modified, engineered, or chemically conjugated. Examples of antigen-binding fragments include Fab, Fab′, (Fab′)₂, Fv, single chain antibodies (e.g., scFv), minibodies, and diabodies. Examples of antibodies that have been modified or engineered include chimeric antibodies, humanized antibodies, and multispecific antibodies (e.g., bispecific antibodies). An example of a chemically conjugated antibody is an antibody conjugated to a toxin moiety.

An antibody molecule may have a heavy chain constant region chosen from, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, the antibody molecule has a light chain constant region chosen from, e.g., the (e.g., human) light chain constant regions of kappa or lambda. The constant region can be altered, e.g., mutated, to modify the properties of the antibody (e.g., to increase or decrease one or more of: Fc receptor binding, antibody glycosylation, the number of cysteine residues, effector cell function, and/or complement function). In one embodiment the antibody has effector function and can fix complement. In other embodiments the antibody does not recruit effector cells or fix complement. In another embodiment, the antibody has reduced or no ability to bind an Fc receptor. For example, it is an isotype or subtype, fragment or other mutant, which does not support binding to an Fc receptor, e.g., it has a mutagenized or deleted Fc receptor binding region.

In certain embodiments, the antibody binds human 5T4 with a K_{D} of 20 nM, 15 nM, 10 nM, 9 nM, 8 nM, 7 nM, 6 nM, 5 nM, 4 nM, 3 nM, 2 nM, 1 nM, 0.75 nM, 0.5 nM, 0.1 nM, 0.075 nM, or 0.05 nM or lower, as measured using standard binding assays, for example, surface plasmon resonance or bio-layer interferometry.

The antibodies disclosed herein may be further optimized (e.g., affinity-matured) to improve biochemical characteristics including affinity and/or specificity, improve biophysical properties including aggregation, stability, precipitation and/or non-specific interactions, and/or to reduce immunogenicity. Affinity-maturation procedures are within ordinary skill in the art. For example, diversity can be introduced into an immunoglobulin heavy chain and/or an immunoglobulin light chain by DNA shuffling, chain shuffling, CDR shuffling, random mutagenesis and/or site-specific mutagenesis.

In certain embodiments, isolated human antibodies contain one or more somatic mutations. In these cases, antibodies can be modified to a human germline sequence to optimize the antibody (i.e., a process referred to as germlining).

Generally, an optimized antibody has at least the same, or substantially the same, affinity for the antigen as the non-optimized (or parental) antibody from which it was derived. Preferably, an optimized antibody has a higher affinity for the antigen when compared to the parental antibody.

In certain embodiments, an antibody can be conjugated to an effector agent such as a small molecule toxin or a radionuclide using standard in vitro conjugation chemistries. If the effector agent is a polypeptide, the antibody can be chemically conjugated to the effector or joined to the effector as a fusion protein. Construction of fusion proteins is within ordinary skill in the art.

The antibody can be conjugated to an effector moiety such as a small molecule toxin or a radionuclide using standard in vitro conjugation chemistries. If the effector moiety is a polypeptide, the antibody can be chemically conjugated to the effector or joined to the effector as a fusion protein. Construction of fusion proteins is within ordinary skill in the art.

In certain embodiments, an antibody may be a bispecific or multispecific antibody. Bispecific antibodies are antibodies that have binding specificities for at least two different epitopes. Exemplary bispecific antibodies may bind to two different epitopes of a single antigen. Other such antibodies may combine a binding site for a first antigen with a binding site for a second antigen. Alternatively, an antigen-specific arm may be combined with an arm that binds to a triggering molecule on a leukocyte, such as a T-cell receptor molecule (e.g., CD3), or Fc receptors for IgG (FcγR), such as FcγRI (CD64), FcγRII (CD32) and Fc gamma RIII (CD16). Bispecific antibodies may also be used to localize cytotoxic agents to infected cells, for example, an antibody may possess an antigen-binding arm and an arm that binds a cytotoxic agent (e.g., saporin, anti-interferon-α, vinca alkaloid, ricin A chain, methotrexate or radioactive isotope hapten). Bispecific antibodies can be prepared as full-length antibodies or antibody fragments. Methods for making bispecific antibodies are known in the art. One example of a bispecific antibody is a bi-specific T-cell engager (BiTE®). A BiTE is a bispecific antibody that directs a T cell's cytotoxic activity to targeted diseased cells. BiTEs are typically fusion proteins including two single-chain variable fragments (scFvs), one of which binds to T cells via the CD3 receptor, and the other to an infected cell via a specific molecule (e.g., 5T4). BiTEs form a link between a T cell and a target cell, causing the T cell to exert cytotoxic activity on the target cell, for example, by producing perforin and granzymes independently of the presence of MHC 1 or co-stimulatory molecules.

Exemplary anti-5T4 antibodies include those described in U.S. Pat. No. 7,531,648, U.S. Patent Publication Nos. US20060088522, US20100173382, US20160185859, US20160304617, US20180162937, US20190194346, and US20190374651, and International (PCT) Publication Nos. WO9855607, WO2001036486, WO2003038098, WO2006031653, WO2007106744, WO2012131527, WO2013068874, WO2014137931, WO2016022939, WO2020076992, and WO2021048423. Further anti-5T4 antibodies are commercially available, for example, from R&D Systems (MN, USA), Lifespan Biosciences, Inc (WA, USA) and Creative Biolabs (NY, USA).

In certain embodiments, an anti-5T4 antibody comprises anti-5T4 variable region sequences fused to the constant region sequences of the murine IgG1/κ antibody C242, for example, the anti-5T4 antibody comprises a chimeric heavy chain, corresponding to residues 1 to 222 of SEQ ID NO: 7, and a chimeric 5T4 Fab light chain corresponding to residues 223-225 of SEQ ID NO: 7. The two protein chains are held together by non-covalent interactions between the Fab heavy and light chains.

### B. Superantigen Conjugates

### 1. Superantigens

Superantigens are bacterial proteins, viral proteins, and human-engineered proteins, capable of activating T lymphocytes, for example, at picomolar concentrations. Superantigens can also activate large subsets of T lymphocytes (T-cells). Superantigens can bind to the major histocompatibility complex I (MHCI) without being processed and, in particular, can bind to conserved regions outside the antigen-binding groove on MHC class II molecules (e.g., on monocytes), avoiding most of the polymorphism in the conventional peptide-binding site. Superantigens can also bind to the Vβ chain of the T-cell receptor (TCR) rather than binding to the hypervariable loops of the T-cell receptor. Examples of bacterial superantigens include, but are not limited to, Staphylococcal enterotoxin (SE), Streptococcus pyogenes exotoxin (SPE), Staphylococcus aureus toxic shock-syndrome toxin (TSST-1), Streptococcal mitogenic exotoxin (SME), Streptococcal superantigen (SSA), Staphylococcal enterotoxin A (SEA), Staphylococcal enterotoxin A (SEB), and Staphylococcal enterotoxin E (SEE).

The polynucleotide sequences encoding many superantigens have been isolated and cloned and superantigens expressed from these or modified (reengineered) polynucleotide sequences have been used in anti-cancer therapy (see, naptumomab estafenatox/ANYARA®, discussed below). Superantigens expressed by these polynucleotide sequences may be wild-type superantigens, modified superantigens, or wild-type or modified superantigens conjugated or fused with targeting moieties. The superantigens may be administered to a mammal, such as a human, directly, for example by injection, or may be delivered, for example, by exposure of blood of a patient to the superantigen outside the body, or, for example, via placing a gene encoding a superantigen inside a mammal to be treated (e.g., via known gene therapy methods and vectors such as, for example, via cells containing, and capable of expressing, the gene) and expressing the gene within the mammal.

Examples of superantigens and their administration to mammals are described in the following U.S. patents and patent applications: U.S. Pat. Nos. 5,858,363, 6,197,299, 6,514,498, 6,713,284, 6,692,746, 6,632,640, 6,632,441, 6,447,777, 6,399,332, 6,340,461, 6,338,845, 6,251,385, 6,221,351, 6,180,097, 6,126,945, 6,042,837, 6,713,284, 6,632,640, 6,632,441, 5,859,207, 5,728,388, 5,545,716, 5,519,114, 6,926,694, 7,125,554, 7,226,595, 7,226,601, 7,094,603, 7,087,235, 6,835,818, 7,198,398, 6,774,218, 6,913,755, 6,969,616, and 6,713,284. U.S. Patent Application Nos. 2003/0157113, 2003/0124142, 2002/0177551, 2002/0141981, 2002/0115190, and 2002/0051765, and PCT International Publication Number WO/03/094846.

### 2. Modified Superantigens

Within the scope of this invention, superantigens may be engineered in a variety of ways, including modifications that retain or enhance the ability of a superantigen to stimulate T lymphocytes, and may, for example, alter other aspects of the superantigen, such as, for example, its seroreactivity or immunogenicity. Modified superantigens include synthetic molecules that have superantigen activity (i.e., the ability to activate subsets of T lymphocytes).

It is contemplated that various changes may be made to the polynucleotide sequences encoding a superantigen without appreciable loss of its biological utility or activity, namely the induction of the T-cell response to result in cytotoxicity of the tumor cells. Furthermore, the affinity of the superantigen for the MHC class II molecule can be decreased with minimal effects on the cytotoxicity of the superantigen. This, for example, can help to reduce toxicity that may otherwise occur if a superantigen retains its wild-type ability to bind MHC class II antigens (as in such a case, class II expressing cells, such as immune system cells, could also be affected by the response to the superantigen).

Techniques for modifying superantigens (e.g., polynucleotides and polypeptides), including for making synthetic superantigens, are well known in the art and include, for example PCR mutagenesis, alanine scanning mutagenesis, and site-specific mutagenesis (see, U.S. Pat. Nos. 5,220,007; 5,284,760; 5,354,670; 5,366,878; 5,389,514; 5,635,377; and 5,789,166).

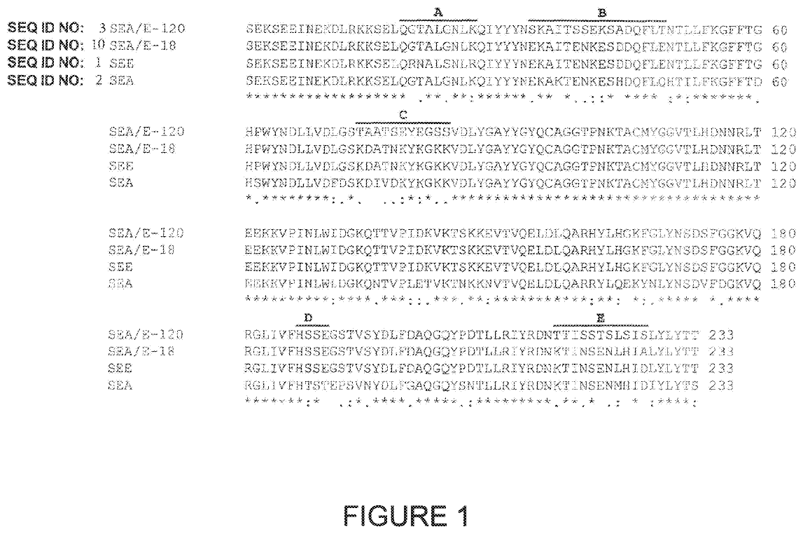

In certain embodiments, a superantigen may be modified such that its seroreactivity is reduced compared to a reference wild-type superantigen, but its ability to activate T-cells is retained or enhanced relative to wild-type. One technique for making such modified superantigens includes substituting certain amino acids in certain regions from one superantigen to another. This is possible because many superantigens, including but not limited to, SEA, SEE, and SED, share sequence homology in certain areas that have been linked to certain functions (Marrack and Kappler (1990) SCIENCE 248(4959): 1066; see also FIG. 1, which shows region of homology between different wild type and engineered superantigens). For example, in certain embodiments of the present invention, a superantigen that has a desired T-cell activation-inducing response, but a non-desired high seroreactivity, is modified such that the resulting superantigen retains its T-cell activation ability but has reduced seroreactivity.

It is known and understood by those of skill in the art that the sera of humans normally contain various titers of antibodies against superantigens. For the staphylococcal superantigens, for instance, the relative titers are TSST-1>SEB>SEC-1>SE3>SEC2>SEA>SED>SEE. As a result, the seroreactivity of, for example, SEE (Staphylococcal enterotoxin E) is lower than that of, for example, SEA (Staphylococcal enterotoxin A). Based on this data, one skilled in the art may prefer to administer a low titer superantigen, such as, for example SEE, instead of a high titer superantigen, such as, for example, SEB (Staphylococcal enterotoxin B). However, as has also been discovered, different superantigens have differing T-cell activation properties relative to one another, and for wild-type superantigens, the best T-cell activating superantigens often also have undesirably high seroreactivity.

These relative titers sometimes correspond to potential problems with seroreactivity, such as problems with neutralizing antibodies. Thus, the use of a low titer superantigen, such as SEA or SEE may be helpful in reducing or avoiding seroreactivity of parenterally administered superantigens. A low titer superantigen has a low seroreactivity as measured, for example, by typical anti-superantigen antibodies in a general population. In some instances it may also have a low immunogenicity. Such low titer superantigens may be modified to retain its low titer as described herein.

Approaches for modifying superantigens can be used to create superantigens that have both the desired T-cell activation properties and reduced seroreactivity, and in some instances also reduced immunogenicity. Given that certain regions of homology between superantigens relate to seroreactivity, it is possible to engineer a recombinant superantigen that has a desired T-cell activation and a desired seroreactivity and/or immunogenicity. Furthermore, the protein sequences and immunological cross-reactivity of the superantigens or staphylococcal enterotoxins are divided into two related groups. One group consists of SEA, SEE and SED. The second group is SPEA, SEC and SEB. Thus, it is possible to select low titer superantigens to decrease or eliminate the cross-reactivity with high titer or endogenous antibodies directed against staphylococcal enterotoxins.

Regions in the superantigens that are believed to play a role in seroreactivity include, for example, Region A, which comprises amino acid residues 20, 21, 22, 23, 24, 25, 26, and 27; Region B, which comprises amino acid residues 34, 35, 36, 37, 38, 39, 40, 41, 42, 43, 44, 45, 46, 47, 48, and 49; Region C, which comprises amino acid residues 74, 75, 76, 77, 78, 79, 80, 81, 82, 83, and 84; Region D, which comprises amino acid residues 187, 188, 189 and 190; and Region E, which comprise the amino acid residues, 217, 218, 219, 220, 221, 222, 223, 224, 225, 226, and 227 (see, U.S. Pat. No. 7,125,554, and FIG. 1 herein). Thus, it is contemplated that these regions can be mutated using, for example amino acid substitution, to produce a superantigen having altered seroreactivity.

Polypeptide or amino acid sequences for the above listed superantigens can be obtained from any sequence data bank, for example Protein Data Bank and/or GenBank. Exemplary GenBank accession numbers include, but are not limited to, SEE is P12993: SEA is P013163: SEB is P01552; SEC1 is P01553: SED is P20723; and SEH is AAA19777.

In certain embodiments of the present invention, the wild-type SEE sequence (SEQ ID NO: 1) or the wild-type SEA sequence (SEQ ID NO: 2) can be modified such that amino acids in any of the identified regions A-E (see, FIG. 1) are substituted with other amino acids. Such substitutions include for example, K79, K81, K83 and D227 or K79, K81, K83, K84 and D227, or, for example, K79E, K81E, K83S and D227S or K79E, K81E, K83S, K84S and D227A. In certain embodiments, the superantigen is SEA/E-120 (SEQ ID NO: 3; see also U.S. Pat. No. 7,125,554). SEA_{D227A} (SEQ ID NO: 4; see also U.S. Pat. No. 7,226,601), or SEA/E-18 (SEQ ID NO: 10; see also U.S. Pat. No. 7,125,554).

A biological functional equivalent of a polynucleotide encoding a naturally occurring or a reference superantigen may comprise a polynucleotide that has been engineered to contain distinct sequences while at the same time retaining the capacity to encode the naturally occurring or reference superantigen. This can be accomplished due to the degeneracy of the genetic code, i.e., the presence of multiple codons, which encode for the same amino acids. In one example, it is possible to introduce a restriction enzyme recognition sequence into a polynucleotide while not disturbing the ability of that polynucleotide to encode a protein. Other polynucleotide sequences may encode superantigens that are different but functionally substantially equivalent in at least one biological property or activity (for example, at least 50%, 60%, 70%, 80%, 90%, 95%, 98% of the biological property or activity, for example, without limitation, the ability to induce a T-cell response that results in cytotoxicity of the tumor cells) to a reference superantigen.

In another example, a polynucleotide may be (and encode) a superantigen functionally equivalent to a reference superantigen even though it may contain more significant changes. Certain amino acids may be substituted for other amino acids in a protein structure without appreciable loss of interactive binding capacity with structures such as, for example, antigen-binding regions of antibodies, binding sites on substrate molecules, receptors, and such like. Furthermore, conservative amino acid replacements may not disrupt the biological activity of the protein, as the resultant structural change often is not one that impacts the ability of the protein to carry out its designed function. It is thus contemplated that various changes may be made in the sequence of genes and proteins disclosed herein, while still fulfilling the goals of the present invention.

Amino acid substitutions may be designed to take advantage of the relative similarity of the amino acid side-chain substituents, for example, their hydrophobicity, hydrophilicity, charge, size, and/or the like. An analysis of the size, shape and/or type of the amino acid side-chain substituents reveals that arginine, lysine and/or histidine are all positively charged residues: that alanine, glycine and/or serine are all a similar size: and/or that phenylalanine, tryptophan and/or tyrosine all have a generally similar shape. Therefore, based upon these considerations, arginine, lysine and/or histidine; alanine, glycine and/or serine; and/or phenylalanine, tryptophan and/or tyrosine; are defined herein as biologically functional equivalents. In addition, it may be possible to introduce non-naturally occurring amino acids. Approaches for making amino acid substitutions with other naturally occurring and non-naturally occurring amino acid are described in U.S. Pat. No. 7,763,253.

In terms of functional equivalents, it is understood that, implicit in the definition of a “biologically functional equivalent” protein and/or polynucleotide, is the concept that there is a limited number of changes that may be made within a defined portion of the molecule while retaining a molecule with an acceptable level of equivalent biological activity. Biologically functional equivalents are thus considered to be those proteins (and polynucleotides) where selected amino acids (or codons) may be substituted without substantially affecting biological function. Functional activity includes the induction of the T-cell response to result in cytotoxicity of the tumor cells.

In addition, it is contemplated that a modified superantigen can be created by substituting homologous regions of various proteins via “domain swapping,” which involves the generation of chimeric molecules using different but, in this case, related polypeptides. By comparing various superantigen proteins to identify functionally related regions of these molecules (see, e.g., FIG. 1), it is possible to swap related domains of these molecules so as to determine the criticality of these regions to superantigen function. These molecules may have additional value in that these “chimeras” can be distinguished from natural molecules, while possibly providing the same function.

In certain embodiments, the superantigen comprises an amino acid sequence that is at least 70%, 75%, 80%, 85%, 90%, 95%, 98%, or 99% identical to the sequence of a reference superantigen selected from SEQ ID NO; 1, SEQ ID NO: 2, SEQ ID NO: 3, and SEQ ID NO: 4, wherein the superantigen optionally retains at least 50%, 60%, 70% 80%, 90%, 95%, 98%, 99%, or 100% of a biological activity or property of the reference superantigen.

In certain embodiments, the superantigen comprises an amino acid sequence that is encoded by a nucleic acid that is at least 70%, 75%, 80%, 85%, 90%, 95%, 98%, or 99% identical to a nucleic acid encoding the superantigen selected from SEQ ID NO: 1, SEQ ID NO: 2, SEQ ID NO: 3, and SEQ ID NO: 4, wherein the superantigen optionally retains at least 50%, 60%, 70% 80%, 90%, 95%, 98%, 99%, or 100% of a biological activity or property of the reference superantigen.

Sequence identity may be determined in various ways that are within the skill in the art, e.g., using publicly available computer software such as BLAST, BLAST-2, ALIGN or Megalign (DNASTAR) software. BLAST (Basic Local Alignment Search Tool) analysis using the algorithm employed by the programs blastp, blastn, blastx, tblastn and tblastx (Karlin et al., (1990) PROC. NATL. ACAD. SCI. USA 87:2264-2268; Altschul, (1993) J. MOL. EVOL. 36, 290-300; Altschul et al., (1997) NUCLEIC ACIDS RES. 25:3389-3402, incorporated by reference) are tailored for sequence similarity searching. For a discussion of basic issues in searching sequence databases see Altschul et al., (1994) NATURE GENETICS 6:119-129, which is fully incorporated by reference. Those skilled in the art can determine appropriate parameters for measuring alignment, including any algorithms needed to achieve maximal alignment over the full length of the sequences being compared. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., (1992) PROC. NATL. ACAD. SCI. USA 89:10915-10919, fully incorporated by reference). Four blastn parameters may be adjusted as follows: Q=10 (gap creation penalty); R=10 (gap extension penalty); wink=1 (generates word hits at every wink.sup.th position along the query); and gapw=16 (sets the window width within which gapped alignments are generated). The equivalent Blastp parameter settings may be Q=9; R=2; wink=1; and gapw=32. Searches may also be conducted using the NCBI (National Center for Biotechnology Information) BLAST Advanced Option parameter (e.g.: -G, Cost to open gap [Integer]: default=5 for nucleotides/11 for proteins; -E, Cost to extend gap [Integer]: default=2 for nucleotides/1 for proteins; -q, Penalty for nucleotide mismatch [Integer]: default=−3; -r, reward for nucleotide match [Integer]: default=1; -e, expect value [Real]: default=10; —W, wordsize [Integer]: default=11 for nucleotides/28 for megablast/3 for proteins; -y, Dropoff (X) for blast extensions in bits: default=20 for blastn/7 for others; -X, X dropoff value for gapped alignment (in bits): default=15 for all programs, not applicable to blastn, and -Z, final X dropoff value for gapped alignment (in bits): 50 for blastn, 25 for others). ClustalW for pairwise protein alignments may also be used (default parameters may include, e.g., Blosum62 matrix and Gap Opening Penalty=10 and Gap Extension Penalty=0.1). A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

### 3. Targeted Superantigens

In order to increase specificity, the superantigen preferably is conjugated to a 5T4-targeting moiety to create a targeted superantigen conjugate. The targeting moiety is a vehicle that can be used to bind superantigen to the cancerous cells, for example, the surface of the cancerous cells. The targeted superantigen conjugate should retain the ability to activate large numbers of T lymphocytes. For example, the targeted superantigen conjugate should activate large numbers of T-cells and direct them to tissues containing the tumor-associated antigen bound to the targeting moiety. In such situations, specific target cells are preferentially killed, leaving the rest of the body relatively unharmed. This type of therapy is desirable, as non-specific anti-cancer agents, such as cytostatic chemotherapeutic drugs, are nonspecific and kill large numbers of cells not associated with tumors to be treated. For example, studies with targeted superantigen conjugates have shown that inflammation with infiltration by cytotoxic T lymphocytes (CTLs) into tumor tissue increases rapidly in response to the first injection of a targeted superantigen (Dohlsten et al. (1995) PROC. NATL. ACAD. SCI. USA 92:9791-9795). This inflammation with infiltration of CTLs into the tumor is one of the major effectors of the anti-tumor therapeutic of targeted superantigens.

Tumor-targeted superantigens represent an immunotherapy against cancer and are therapeutic fusion proteins containing a targeting moiety conjugated to a superantigen (Dohlsten et al. (1991) PROC. NATL. ACAD. SCI. USA 88:9287-9291: Dohlsten et al. (1994) PROC. NATL. ACAD. SCI. USA 91:8945-8949).

The targeting moiety can in principle be any structure that is able to bind to 5T4. The targeting moiety can be selected from antibodies, including antigen binding fragments thereof, soluble T-cell receptors, growth factors, interleukins (e.g., interleukin-2), hormones, etc.

In certain preferred embodiments, the targeting moiety is an antibody (e.g., Fab. F(ab)₂, Fv, single chain antibody, etc.). Exemplary anti-5T4 antibodies include those described in U.S. Pat. No. 7,531,648, U.S. Patent Publication Nos. US20060088522, US20100173382, US20160185859, US20160304617, US20180162937, US20190194346, and US20190374651, and International (PCT) Publication Nos. WO9855607, WO2001036486, WO2003038098, WO2006031653, WO2007106744, WO2012131527, WO2013068874, WO2014137931, WO2016022939, WO2020076992, and WO2021048423. Further anti-5T4 antibodies are commercially available, for example, from R&D Systems (MN, USA), Lifespan Biosciences, Inc (WA, USA) and Creative Biolabs (NY, USA).

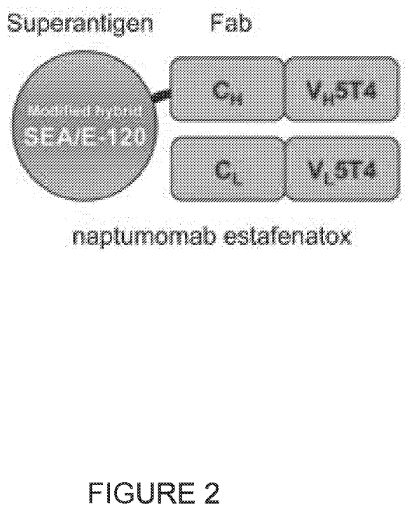

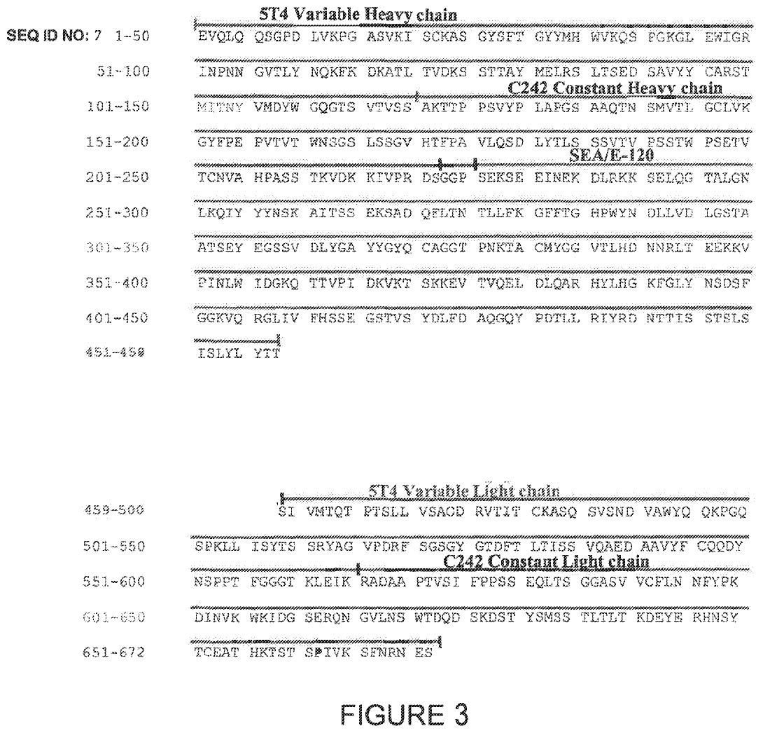

Examples of tumor targeted superantigens that can be used in the present invention include 5T4Fab-SEA_{D227A} (SEQ ID NO: 6) and 5T4Fab-SEA/E-120 (SEQ ID NO: 7, see FIG. 2 and FIG. 3).

In a preferred embodiment, a preferred conjugate is a superantigen conjugate known as naptumomab estafenatox/ANYARA®, which is the fusion protein of the Fab fragment of an anti-5T4 antibody and the SEA/E-120 superantigen (see, FIG. 2). Naptumomab estafenatox/ANYARA® comprises two protein chains that cumulatively include an engineered Staphylococcal enterotoxin superantigen (SEA/E-120) and a targeting 5T4 Fab comprising modified 5T4 variable region sequences fused to the constant region sequences of the murine IgG1/κ antibody C242. With reference to FIG. 3, the first protein chain comprises residues 1 to 458 of SEQ ID NO: 7 (see also, SEQ ID NO: 8), and includes a chimeric 5T4 Fab heavy chain, corresponding to residues 1 to 222 of SEQ ID NO: 7, and the SEA/E-120 superantigen, corresponding to residues 226 to 458 of SEQ ID NO: 7, covalently linked via a GGP tripeptide linker, corresponding to residues 223-225 of SEQ ID NO: 7. The second chain comprises residues 459 to 672 of SEQ ID NO: 7 (see also, SEQ ID NO: 9) and includes a chimeric 5T4 Fab light chain. The two protein chains are held together by non-covalent interactions between the Fab heavy and light chains. Residues 1-458 of SEQ ID NO: 7 correspond to residues 1-458 of SEQ ID NO: 8, and residues 459-672 of SEQ ID NO: 7 correspond to residues 1-214 of SEQ ID NO: 9. Naptumomab estafenatox/ANYARA® comprises the proteins of SEQ ID NOS: 8 and 9 held together by non-covalent interactions between the Fab heavy and Fab light chains. Naptumomab estafenatox/ANYARA® induces T-cell mediated killing of cancer cells at concentrations around 10 pM and the superantigen component of the conjugate has been engineered to have low binding to human antibodies and MHC Class II.

It is contemplated that other antibody-based targeting moieties can be designed, modified, expressed, and purified using techniques known in the art and discussed in more detail below.

Another type of targeting moiety includes a soluble T-cell receptor (TCR). Some forms of soluble TCR may contain either only extracellular domains or extracellular and cytoplasmic domains. Other modifications of the TCR may also be envisioned to produce a soluble TCR in which the transmembrane domains have been deleted and/or altered such that the TCR is not membrane bound as described in U.S. Publication Application Nos. U.S. 2002/119149, U.S. 2002/0142389, U.S. 2003/0144474, and U.S. 2003/0175212, and International Publication Nos. WO2003020763, WO9960120, and WO9960119.

The targeting moiety can be conjugated to the superantigen by using either recombinant techniques or chemically linking of the targeting moiety to the superantigen.

It is contemplated that a gene encoding a superantigen linked directly or indirectly (for example, via an amino acid containing linker) to a targeting moiety can be created and expressed using conventional recombinant DNA technologies. For example, the amino terminal of a modified superantigen can be linked to the carboxy terminal of a targeting moiety or vice versa. For antibodies, or antibody fragments that may serve as targeting moieties, either the light or the heavy chain may be utilized for creating a fusion protein. For example, for a Fab fragment, the amino terminus of the modified superantigen can be linked to the first constant domain of the heavy antibody chain (CH₁). In some instances, the modified superantigen can be linked to a Fab fragment by linking the VH and VL domain to the superantigen. Alternatively, a peptide linker can be used to join the superantigen and targeting moiety together. When a linker is employed, the linker preferably contains hydrophilic amino acid residues, such as Gln, Ser, Gly, Glu, Pro, His and Arg. Preferred linkers are peptide bridges consisting of 1-10 amino acid residues, more particularly, 3-7 amino acid residues. An exemplary linker is the tripeptide-GlyGlyPro-. These approaches have been used successfully in the design and manufacture of the naptumomab estafenatox/ANYARA® superantigen conjugate.

It is also contemplated that the superantigen may be linked to the targeting moiety via a chemical linkage. Chemical linkage of the superantigen to the targeting moiety may require a linker, for example, a peptide linker. The peptide linker preferably is hydrophilic and exhibits one or more reactive moieties selected from amides, thioethers, disulfides etc (see, U.S. Pat. Nos. 5,858,363, 6,197,299, and 6,514,498). It is also contemplated that the chemical linkage can use homo- or heterobifunctional crosslinking reagents. Chemical linking of a superantigen to a targeting moiety often utilizes functional groups (e.g., primary amino groups or carboxy groups) that are present in many positions in the compounds.

Examples of production systems for superantigens are found, for example, in U.S. Pat. No. 6,962,694.

### C. Immune Cells

Immune cells include, e.g., lymphocytes, such as B-cells and T-cells, natural killer cells (NK-cells), natural killer T-cells (NKT-cells), myeloid cells, such as monocytes, macrophages, eosinophils, mast cells, basophils, and granulocytes.

In certain embodiments, the immune cell is a T-cell, which can be, for example, a cultured T-cell, e.g., a primary T-cell, or a T-cell from a cultured T-cell line, e.g., Jurkat, SupTi, etc., or a T-cell obtained from a mammal, for example, from a subject to be treated. If obtained from a mammal, the T-cell can be obtained from numerous sources, including but not limited to blood, bone marrow, lymph node, the thymus, or other tissues or fluids. T-cells can also be enriched or purified. The T-cell can be any type of T-cell and can be of any developmental stage, including but not limited to, CD4+/CD8+ double positive T-cells, CD4+ helper T-cells, e.g., Th1 and Th2 cells, CD4+ T-cells, CD8+ T-cells (e.g., cytotoxic T-cells), tumor infiltrating lymphocytes (TILs), memory T-cells (e.g., central memory T-cells and effector memory T-cells), naive T-cells, and the like. The cells (e.g., the T-cells) can include autologous cells derived from a subject to be treated, or alternatively allogenic cells derived from a donor.

In certain embodiments, the T-cell binds 5T4 through a T-cell receptor. The T-cell receptor may be an endogenous or a recombinant T-cell receptor. T-cell receptors comprise two chains referred to as the α- and β-chains, that combine on the surface of a T-cell to form a heterodimeric receptor that can recognize MHC-restricted antigens. Each of α- and β-chain comprises two regions, a constant region and a variable region. Each variable region of the α- and β-chains defines three loops, referred to as complementary determining regions (CDRs) known as CDR₁, CDR₂, and CDR₃ that confer the T-cell receptor with antigen binding activity and binding specificity.

In certain embodiments, the immune cell, e.g., T-cell or NKT-cell, binds to 5T4 through a chimeric antigen receptor (CAR), i.e., the T-cell or NKT-cell comprises an exogenous nucleotide sequence encoding a CAR. As used herein, the terms “chimeric antigen receptor,” or “CAR,” refer to any artificial receptor including an antigen-specific binding moiety and one or more signaling chains derived from an immune receptor. CARs can comprise a single chain fragment variable (scFv) of an antibody specific for an antigen coupled via hinge and transmembrane regions to cytoplasmic domains of T-cell signaling molecules (e.g. a T-cell costimulatory domain (e.g., from CD28, CD137, OX40, ICOS, or CD27) in tandem with a T-cell triggering domain (e.g. from CD3ζ) and/or to cytoplasmic domains of NK-cell signaling molecules (e.g. DNAX-activation protein 12 (DAP12)). A T-cell expressing a chimeric antigen receptor is referred to as a CAR T-cell, an NK-cell expressing a chimeric antigen receptor is referred to as a CAR NK-cell, and an NKT-cell expressing a chimeric antigen receptor is referred to as a CAR NKT-cell.

Exemplary 5T4-targeted CAR T-cells are described U.S. Patent Publication Nos. US20200407461 and US20200010560, and International (PCT) Publication No WO2016022939.

CAR T-cells may be generated using methods known in the art. T-cells can be obtained from a number of sources, including peripheral blood mononuclear cells, bone marrow, lymph node tissue, cord blood, thymus tissue, tissue from a site of infection, ascites, pleural effusion, spleen tissue, tumors, and T-cell lines. For example, T-cells can be obtained from a unit of blood collected from a subject using any number of techniques known to the skilled artisan, such as Ficoll™ separation. In certain embodiments, cells from the circulating blood of an individual are obtained by apheresis. The apheresis product typically contains lymphocytes, including T-cells, monocytes, granulocytes, B cells, other nucleated white blood cells, red blood cells, and platelets. Cells collected by apheresis may be washed to remove the plasma fraction and to place the cells in an appropriate buffer or media for subsequent processing steps. For example, the cells may be washed with phosphate buffered saline (PBS). After washing, the cells may be resuspended in a variety of biocompatible buffers, such as, for example. Ca²⁺-free or Mg²⁺-free PBS, PlasmaLyte A, or other saline and/or buffer solutions. T-cells may also be isolated from peripheral blood lymphocytes by lysing red blood cells and depleting monocytes, for example, by centrifugation through a PERCOLL™ gradient or by counterflow centrifugal elutriation. A specific subpopulation of T-cells, such as CD3+, CD28+, CD4+, CD8+, CD45RA+, and CD45RO+ T-cells, can be further isolated by positive or negative selection techniques. For example, in one embodiment, T-cells are isolated by incubation with anti-CD3/anti-CD28-conjugated beads, such as DYNABEADS® M-450 CD3/CD28 (Thermo Fisher Scientific), for a time period sufficient for positive selection of the desired T-cells.

T-cells may be engineered to express CARs by methods known in the art. Generally, a poly nucleotide vector is constructed that encodes the CAR and the vector is transfected or transduced into a population of T-cells. For example, a nucleotide sequence encoding a CAR can be delivered into cells using a retroviral or lentiviral vector An exemplary retroviral vector includes, but is not limited to, the vector backbone pMSGV1-CD8-28BBZ, which is derived from pMSGV (murine stem cell virus-based splice-gag vector). For other exemplary lentiviral vectors see, for example, Dull et at, (1998) J. VIROL. 72:8463-8471, and U.S. Pat. Nos. 5,994,136, 6,682,907, 7,629,153, 8,329,462, 8,748,169, 9,101,584. Retroviral transduction may be performed using known techniques, such as that of Johnson et al. (2009) BLOOD 114, 535-546. The surface expression of a CAR on transduced T-cells may be determined, for example, by flow cytometry. A nucleotide sequence encoding a CAR can also be determined into cells using in vitro transcribed mRNA.

T-cells and/or T-cells engineered to express CARs can be activated and expanded generally using methods as described, for example, in U.S. Pat. Nos. 6,352,694; 6,534,055, 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005. Generally, T-cells are expanded by contact with an agent that stimulates a CD3/TCR complex associated signal and a ligand that stimulates a co-stimulatory molecule on the surface of the T-cells. For example, T-cell populations may be stimulated by contact with an anti-CD3 antibody, anti-CD28 antibody, an anti-CD2 antibody, or a protein kinase C activator (e.g., bry ostatin) and/or a calcium ionophore.

Further methods for manufacturing CAR T-cells are described, for example, in Levine et al. (2016) MOL. THER. METHODS CLIN. DEV. 4:92-101.

Examples of systems for expressing CARs or regulating expression of CARs include “ON-Switch” CARs (Wu et al. (2015) SCIENCE 350: aab4077), combinatorial activation systems (Fedorov et al. (2014) CANCER JOURNAL 20:160-165; Kloss et al. (2013) NATURE BIOTECHNOLOGY 31: 71-75), doxycycline-inducible CARs (Sakemura et al. (2016) CANCER IMMUNOL. RES. 4:658-668), antibody-inducible CARs (Hill et al. (2018) NATURE CHEMICAL BIOLOGY 14:112-117), kill switches (Di Stasi et al. (2011) N. ENGL. J. MED. 365:1673-1683 (2011); Budde et al. (2013) PLOS ONE 8: e82742), pause switches (Wei et al. (2012) NATURE 488: 384-388), tunable receptor systems (Ma et al. (2016) PROC. NATL. ACAD. SCI. USA 113: E450-458; Rodgers et al. (2016) PROC. NATL. ACAD. SCI. USA 113: E459-468; Kudo et al. (2014) CANCER RES. 74: 93-103), and proliferation switches (Chen et al. (2010) PROC. NATL. ACAD. SCI. USA 107, 8531-8536).

### D. Expression Methods

A protein of interest, e.g., an antibody, a superantigen conjugate, a chimeric antigen receptor, and/or a T-cell receptor subunit, may be expressed in a host cell of interest by incorporating a gene encoding the protein of interest into an appropriate expression vector.

Host cells can be genetically engineered, for example, by transformation or transfection technologies, to incorporate nucleic acid sequences and express the superantigen. Introduction of nucleic acid sequences into the host cell can be affected by calcium phosphate transfection, DEAE-dextran mediated transfection, microinjection, cationic lipid-mediated transfection, electroporation, transduction, scrape loading, ballistic introduction, infection or other methods. Such methods are described in many standard laboratory manuals, such as, Davis et al. (1986) BASIC METHODS IN MOLECULAR BIOLOGY and Sambrook, et al. (1989) MOLECULAR CLONING: A LABORATORY MANUAL, 2nd Ed., Cold Spring Harbor Laboratory Press, Cold Spring Harbor, N.Y.

Representative examples of appropriate host cells include bacterial cells, such as streptococci, staphylococci, E. coli, Streptomyces and Bacillus subtilis cells; fungal cells, such as yeast cells and aspergillus cells; insect cells such as Drosophila S2 and Spodoptera Sf9 cells, mammalian cells such as CHO, COS, HeLa, C127, 3T3, BHK, HEK-293 and Bowes melanoma cells.

When recombinant DNA technologies are employed a protein of interest may be expressed using standard expression vectors and expression systems. The expression vectors, which have been genetically engineered to contain the nucleic acid sequence encoding the superantigen, are introduced (e.g., transfected) into host cells to produce the superantigen (see, e.g. Dohlsten et al. (1994), Forsberg et al. (1997) J. BIOL. CHEM. 272:12430-12436, Erlandsson et al. (2003) J. MOL. BIOL. 333:893-905 and WO2003002143).

As used herein, “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes), retrotransposons (e.g. piggyback, sleeping beauty), and viruses (e.g., lentiviruses, retroviruses, adenoviruses, and adeno-associated viruses) that incorporate the recombinant polynucleotide of interest.

In certain embodiments, the expression vector is a viral vector. The term “virus” is used herein to refer to an obligate intracellular parasite having no protein-synthesizing or energy-generating mechanism. Exemplary viral vectors include retroviral vectors (e.g., lentiviral vectors), adenoviral vectors, adeno-associated viral vectors, herpes viruses vectors, epstein-barr virus (EBV) vectors, polyonmavirus vectors (e.g. simian vacuolating virus 40 (SV40) vectors), poxvirus vectors, and pseudotype virus vectors.

The virus may be an RNA virus (having a genome that is composed of RNA) or a DNA virus (having a genome composed of DNA). In certain embodiments, the viral vector is a DNA virus vector. Exemplary DNA viruses include parvoviruses (e.g., adeno-associated viruses), adenoviruses, asfarviruses, herpesviruses (e.g., herpes simplex virus 1 and 2 (HSV-1 and HSV-2), epstein-barr virus (EBV), cytomegalovirus (CMV)), papillomoviruses (e.g., HPV), polyomaviruses (e.g., simian vacuolating virus 40 (SV40)), and poxviruses (e.g., vaccinia virus, cowpox virus, smallpox virus, fowlpox virus, sheeppox virus, myxoma virus). In certain embodiments, the viral vector is an RNA virus vector. Exemplary RNA viruses include bunyaviruses (e.g., hantavirus), coronaviruses, flaviviruses (e.g., yellow fever virus, west nile virus, dengue virus), hepatitis viruses (e.g., hepatitis A virus, hepatitis C virus, hepatitis E virus), influenza viruses (e.g., influenza virus type A, influenza virus type B, influenza virus type C), measles virus, mumps virus, noroviruses (e.g., Norwalk virus), poliovirus, respiratory syncytial virus (RSV), retroviruses (e.g., human immunodeficiency virus-1 (HIV-1)) and toroviruses.

In certain embodiments, the expression vector comprises a regulatory sequence or promoter operably linked to the nucleotide sequence encoding the protein of interest, e.g., a superantigen conjugate, a chimeric antigen receptor, and/or a T-cell receptor subunit. The term “operably linked” refers to a linkage of polynucleotide elements in a functional relationship. A nucleic acid sequence is “operably linked” when it is placed into a functional relationship with another nucleic acid sequence. For instance, a promoter or enhancer is operably linked to a gene if it affects the transcription of the gene. Operably linked nucleotide sequences are typically contiguous. However, as enhancers generally function when separated from the promoter by several kilobases and intronic sequences may be of variable lengths, some polynucleotide elements may be operably linked but not directly flanked and may even function in trans from a different allele or chromosome.

Exemplary promoters which may be employed include, but are not limited to, the retroviral LTR, the SV40 promoter, the human cytomegalovirus (CMV) promoter, the U6 promoter, or any other promoter (e.g., cellular promoters such as eukaryotic cellular promoters including, but not limited to, the histone, pol III, and D-actin promoters). Other viral promoters which may be employed include, but are not limited to, adenovirus promoters, TK promoters, and B19 parvovirus promoters.

In certain embodiments, a promoter is an inducible promoter. The use of an inducible promoter allows for expression of an operatively linked polynucleotide sequence to be turned on or off when desired. In certain embodiments, the promoter is induced in the presence of an exogenous molecule or activity, e.g., a metallothionine promoter, a glucocorticoid promoter, a progesterone promoter, and a tetracycline promoter. In certain embodiments, the promoter is induced in the tumor microenvironment, e.g., an IL-2 promoter, a NFAT promoter, a cell surface protein promoter (e.g., a CD69 promoter or a PD-1 promoter), a cytokine promoter (e.g., a TNF promoter), a cellular activation promoter (e.g., a CTLA4, OX40, or CD40L promoter), or a cell surface adhesion protein promoter (e.g., a VLA-1 promoter).

In certain embodiments, a promoter mediates rapid, sustained expression, measured in days (e.g., a CD69 promoter). In certain embodiments, a promoter mediates delayed, late-inducible expression (e.g., a VLA1 promoter). In certain embodiments, a promoter mediates rapid, transient expression (e.g., a TNF promoter, an immediate early response gene promoter and others).

The selection of a promoter, e.g., strong, weak, inducible, tissue-specific, developmental-specific, having specific kinetics of activation (e.g., early and/or late activation), and/or having specific kinetics of expression of an induced gene (e.g., short or long expression) is within the ordinary skill of the artisan and will be apparent to those skilled in the art from the teachings contained herein.

In certain embodiments, a protein of interest, e.g., an antibody or a superantigen conjugate, are purified prior to use, which can be accomplished using a variety of purification protocols. The protein of interest may be purified using chromatographic and electrophoretic techniques to achieve partial or complete purification (or purification to homogeneity). Analytical methods particularly suited to the preparation of a pure peptide are ion-exchange chromatography, size exclusion chromatography; affinity chromatography; polyacrylamide gel electrophoresis; isoelectric focusing. The term “purified” as used herein, is intended to refer to a composition, isolatable from other components, wherein the macromolecule (e.g., protein) of interest is purified to any degree relative to its original state. Generally, the terms “purified” refer to a macromolecule that has been subjected to fractionation to remove various other components, and which substantially retains its biological activity. The term “substantially purified” refers to a composition in which the macromolecule of interest forms the major component of the composition, such as constituting about 50%, about 60%, about 70%, about 80%, about 90%, about 95% or more of the content of the composition.

Various methods for quantifying the degree of purification of the protein are known to those of skill in the art, including, for example, determining the specific activity of an active fraction, and assessing the amount of a given protein within a fraction by SDS-PAGE analysis, High Performance Liquid Chromatography (HPLC), or any other fractionation technique. Various techniques suitable for use in protein purification include, for example, precipitation with ammonium sulfate, PEG, antibodies and the like or by heat denaturation, followed by centrifugation; chromatography steps such as ion exchange, gel filtration, reverse phase, hydroxyapatite, affinity chromatography; isoelectric focusing; gel electrophoresis; and combinations of such and other techniques. It is contemplated that the order of conducting the various purification steps may be changed, or that certain steps may be omitted, and still result in a suitable method for the preparation of a substantially purified protein or peptide.

### III. Pharmaceutical Compostions

For therapeutic use, a 5T4-targeting agent preferably is combined with a pharmaceutically acceptable carrier. The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable carrier” as used herein refers to buffers, carriers, and excipients suitable for use in contact with the tissues of human beings and animals without excessive toxicity, irritation, allergic response, or other problem or complication, commensurate with a reasonable benefit/risk ratio. Pharmaceutically acceptable carriers include any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see, e.g., Martin, Remington's Pharmaceutical Sciences, 15th Ed., Mack Publ. Co., Easton, PA [1975]. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

In certain embodiments, a pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents: surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants (See Remington's Pharmaceutical Sciences, 18th ed. (Mack Publishing Company, 1990).

In certain embodiments, a pharmaceutical composition may contain nanoparticles, e.g., polymeric nanoparticles, liposomes, or micelles (See Anselmo et al. (2016) BIOENG. TRANSL. MED. 1: 10-29).

In certain embodiments, a pharmaceutical composition may contain a sustained- or controlled-delivery formulation. Techniques for formulating sustained- or controlled-delivery means, such as liposome carriers, bio-erodible microparticles or porous beads and depot injections, are also known to those skilled in the art. Sustained-release preparations may include, e.g., porous polymeric microparticles or semipermeable polymer matrices in the form of shaped articles, e.g., films, or microcapsules. Sustained release matrices may include polyesters, hydrogels, polylactides, copolymers of L-glutamic acid and gamma ethyl-L-glutamate, poly (2-hydroxyethyl-inethacrylate), ethylene vinyl acetate, or poly-D(−)-3-hydroxybutyric acid. Sustained release compositions may also include liposomes that can be prepared by any of several methods known in the art.

Pharmaceutical compositions containing a 5T4-targeting agent disclosed herein can be presented in a dosage unit form and can be prepared by any suitable method. A pharmaceutical composition should be formulated to be compatible with its intended route of administration. Examples of routes of administration are intravenous (IV), intramuscular, intradermal, inhalation, transdermal, topical, transmucosal, intrathecal and rectal administration. In certain embodiments, a pharmaceutical composition containing a 5T4-targeting agent disclosed herein is administered by IV infusion. Alternatively, the agents may be administered locally rather than systemically, for example, via injection of the agent or agents directly into the site of action, often in a depot or sustained release formulation. In certain embodiments, a pharmaceutical composition containing a 5T4-targeting agent disclosed herein is administered by intratumoral injection.

Useful formulations can be prepared by methods known in the pharmaceutical art. For example, see Remington's Pharmaceutical Sciences, 18th ed. (Mack Publishing Company, 1990). Formulation components suitable for parenteral administration include a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor ELTM (BASF, Parsippany, NJ) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyethylene glycol), and suitable mixtures thereof.

Pharmaceutical formulations preferably are sterile. Sterilization can be accomplished by any suitable method, e.g., filtration through sterile filtration membranes. Where the composition is lyophilized, filter sterilization can be conducted prior to or following lyophilization and reconstitution.

In certain embodiments, pharmaceutical compositions may comprise, for example, at least about 0.1% of an active compound. In other embodiments, the active compound may comprise between about 2% to about 75% of the weight of the unit, or between about 25% to about 60%, for example, and any range derivable therein. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable. Such determinations are known and used by those of skill in the art.

The active agents are administered in an amount or amounts effective to decrease, reduce, inhibit or otherwise abrogate the growth or proliferation of cancer cells, induce apoptosis, inhibit angiogenesis of a cancer or tumor, inhibit metastasis, or induce cytotoxicity in cells. The effective amount of active compound(s) used to practice the present invention for therapeutic treatment of cancer varies depending upon the manner of administration, the age, body weight, and general health of the subject. These terms include synergistic situations wherein a single agent alone may act weakly or not at all, but when combined with another agent, for example, but not limited to, via sequential dosage, the two or more agents act to produce a synergistic result.

Generally, a therapeutically effective amount of active component is in the range of 0.1 mg/kg to 100 mg/kg, e.g., 1 mg/kg to 100 mg/kg, 1 mg/kg to 10 mg/kg. The amount administered will depend on variables such as the type and extent of disease or indication to be treated, the overall health of the patient, the in vivo potency of the antibody, the pharmaceutical formulation, and the route of administration. The initial dosage can be increased beyond the upper level in order to rapidly achieve the desired blood-level or tissue-level. Alternatively, the initial dosage can be smaller than the optimum, and the daily dosage may be progressively increased during the course of treatment. Human dosage can be optimized, e.g., in a conventional Phase I dose escalation study designed to run from 0.5 mg/kg to 20 mg/kg. Dosing frequency can vary, depending on factors such as route of administration, dosage amount, serum half-life of the antibody, and the disease being treated. Exemplary dosing frequencies are once per day, once per week and once every two weeks. A preferred route of administration is parenteral, e.g., intravenous infusion. In certain embodiments, a 5T4-targeting agent is lyophilized, and then reconstituted in buffered saline, at the time of administration.

In certain embodiments, when the 5T4-targeting agent is an isolated, naturally occurring or engineered immune cell, e.g., T-cell, a dose is in the range of, e.g., 10⁵ to 10⁹ cells/kg, 10⁵ to 10⁸ cells/kg, 10⁵ to 10⁷ cells/kg, 10⁵ to 10⁶ cells/kg, 10⁶ to 10⁹ cells/kg, 10⁶ to 10⁸ cells/kg, 10⁶ to 10⁷ cells/kg, 10⁷ to 10⁹ cells/kg, 10⁷ to 10⁸ cells/kg, or 10⁸ to 10⁹ cells/kg, or 10⁶ to 10⁷ total cells, 10⁶ to 10¹⁰ total cells, 10⁶ to 10⁹ total cells, 10⁶ to 10⁸ total cells, 10⁶ to 10⁷ total cells, 10⁷ to 10¹¹ total cells, 10⁷ to 10¹⁰ total cells, 10⁷ to 10⁹ total cells, 10⁷ to 10⁸ total cells, 10⁸ to 10¹⁰ total cells, 10⁸ to 10¹⁰ total cells 10⁸ to 10⁹ total cells, 10⁹ to 10¹¹ total cells, 10⁹ to 10¹⁰ total cells, or 10¹⁰ to 10¹¹ total cells. The amount administered will depend on variables such as the type and extent of disease or indication to be treated, the overall health of the patient, the in vivo potency of the active agent, the pharmaceutical formulation, and the route of administration. Progress can be monitored by periodic assessment.

In certain non-limiting examples, a dose of a 5T4-targeting agent may also comprise from about 1 microgram/kg/body weight, about 5 microgram/kg/body weight, about 10 microgram/kg/body weight, about 15 microgram/kg/body weight, about 20 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1,000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, about 1 microgram/kg/body weight to about 100 milligram/kg/body weight. Other exemplary dosage ranges, range from about 1 microgram/kg/body weight to about 1,000 microgram/kg/body weight, from about 1 microgram/kg/body weight to about 100 microgram/kg/body weight, from about 1 microgram/kg/body weight to about 75 microgram/kg/body weight, from about 1 microgram/kg/body weight to about 50 microgram/kg/body weight, from about 1 microgram/kg/body weight to about 40 microgram/kg/body weight, from about 1 microgram/kg/body weight to about 30 microgram/kg/body weight, from about 1 microgram/kg/body weight to about 20 microgram/kg/body weight, from about 1 microgram/kg/body weight to about 15 microgram/kg/body weight, from about 1 microgram/kg/body weight to about 10 microgram/kg/body weight, from about 5 microgram/kg/body weight to about 1,000 microgram/kg/body weight, from about 5 microgram/kg/body weight to about 100 microgram/kg/body weight, from about 5 microgram/kg/body weight to about 75 microgram/kg/body weight, from about 5 microgram/kg/body weight to about 50 microgram/kg/body weight, from about 5 microgram/kg/body weight to about 40 microgram/kg/body weight, from about 5 microgram/kg/body weight to about 30 microgram/kg/body weight, from about 5 microgram/kg/body weight to about 20 microgram/kg/body weight, from about 5 microgram/kg/body weight to about 15 microgram/kg/body weight, from about 5 microgram/kg/body weight to about 10 microgram/kg/body weight, from about 10 microgram/kg/body weight to about 1,000 microgram/kg/body weight, from about 10 microgram/kg/body weight to about 100 microgram/kg/body weight, from about 10 microgram/kg/body weight to about 75 microgram/kg/body weight, from about 10 microgram/kg/body weight to about 50 microgram/kg/body weight, from about 10 microgram/kg/body weight to about 40 microgram/kg/body weight, from about 10 microgram/kg/body weight to about 30 microgram/kg/body weight, from about 10 microgram/kg/body weight to about 20 microgram/kg/body weight, from about 10 microgram/kg/body weight to about 15 microgram/kg/body weight, from about 15 microgram/kg/body weight to about 1,000 microgram/kg/body weight, from about 15 microgram/kg/body weight to about 100 microgram/kg/body weight, from about 15 microgram/kg/body weight to about 75 microgram/kg/body weight, from about 15 microgram/kg/body weight to about 50 microgram/kg/body weight, from about 15 microgram/kg/body weight to about 40 microgram/kg/body weight, from about 15 microgram/kg/body weight to about 30 microgram/kg/body weight, from about 15 microgram/kg/body weight to about 20 microgram/kg/body weight, from about 20 microgram/kg/body weight to about 1,000 microgram/kg/body weight, from about 20 microgram/kg/body weight to about 100 microgram/kg/body weight, from about 20 microgram/kg/body weight to about 75 microgram/kg/body weight, from about 20 microgram/kg/body weight to about 50 microgram/kg/body weight, from about 20 microgram/kg/body weight to about 40 microgram/kg/body weight, from about 20 microgram/kg/body weight to about 30 microgram/kg/body weight, etc., can be administered, based on the numbers described above.

In certain embodiments, for example, administration of a 5T4-targeting agent, the effective amount or dose of the 5T4-targeting agent that is administered is an amount in the range of 0.01 to 500 μg/kg body weight of the subject, for example, 0.1-500 μg/kg body weight of the subject, and, for example, 1-100 μg/kg body weight of the subject.

The compositions described herein may be administered locally or systemically. Administration will generally be parenteral administration. In a preferred embodiment, the pharmaceutical composition is administered subcutaneously and in an even more preferred embodiment intravenously. Preparations for parenteral administration include sterile aqueous or non-aqueous solutions, suspensions, and emulsions.

### IV. Therapeutic Uses

The compositions and methods disclosed herein can be used to treat various forms of glioblastoma in a subject or inhibit glioblastoma growth in a subject. The invention provides a method of treating glioblastoma in a subject. The method comprises administering to the subject an effective amount of a disclosed 5T4-targeting agent, either alone or in a combination with another therapeutic agent to treat the glioblastoma in the subject. For example, the disclosed 5T4-targeting agent can be administered to the subject to slow the growth rate of cancer cells, reduce the incidence or number of metastases, reduce tumor size, inhibit tumor growth, reduce the blood supply to a tumor or cancer cells, promote an immune response against cancer cells or a tumor, prevent or inhibit the progression of cancer, for example, by at least 40%, 50%, 60%, 70%, 80%, 90%, 95%, 98%, 99%, or 100%. Alternatively, the 5T4-targeting agent can be administered to the subject so as to treat the cancer, for example, to increase the lifespan of a subject with cancer, for example, by 3 months, 6 months, 9 months. 12 months, 1 year, 5 years, or 10 years.

It is contemplated that a number of glioblastomas may be treated using the methods and compositions described herein, including but not limited to proneural glioblastoma, neural glioblastoma, classical glioblastoma, mesenchymal glioblastoma, primary glioblastoma, secondary glioblastoma, grade I glioblastoma, grade II glioblastoma, grade III glioblastoma, and/or grade IV glioblastoma.

It is contemplated that 5T4 expression may be measured in a sample, e.g., a sample from a subject. 5T4 can be detected in tissue, body fluid and/or cell samples from subjects of interest using techniques known in the art. The body fluid sample can be, for example, blood, serum or plasma. The tissue sample can be, for example, tumor tissue. The cell sample can be, for example, a cancer cell sample. It is understood that the tumor tissue or any of the samples may be preserved or processed using techniques known in the art, e.g., formalin-fixed, paraffin-embedded sections.

Preferably, patients to be treated will have adequate bone marrow function (defined as a peripheral absolute granulocyte count of >2,000/mm³ and a platelet count of 100,000/mm³), adequate liver function (bilirubin<1.5 mg/dl) and adequate renal function (creatinine<1.5 mg/dl).

Treatment regimens may vary, and often depend on tumor size, tumor location, disease progression, and health and age of the patient. Certain types of tumor may require more aggressive treatment protocols, but at the same time, the patients may be unable to tolerate more aggressive treatment regimens. The clinician may often be best suited to make such decisions based on his or her skill in the art and the known efficacy and toxicity (if any) of the therapeutic formulations.

A typical course of treatment, for a primary tumor or a post-excision tumor bed, may involve multiple doses. Typical primary tumor treatment may involve a 6-dose application over a two-week period. The two-week regimen may be repeated one, two, three, four, five, six or more times. During a course of treatment, the need to complete the planned dosings may be re-evaluated.

Immunotherapy with a superantigen conjugate often results in rapid (within hours) and powerful polyclonal activation of T lymphocytes. A superantigen conjugate treatment cycle may include 4 to 5 daily intravenous superantigen conjugate drug injections. Such treatment cycles can be given in e.g., 4 to 6 week intervals. The inflammation with infiltration of CTLs into the tumor is one of the major effectors of the anti-tumor therapeutic superantigens. After a short period of massive activation and differentiation of CTLs, the T-cell response declines rapidly (within 4-5 days) back to base line levels. Thus, the period of lymphocyte proliferation, during which cytostatic drugs may interfere with superantigen treatment is short and well-defined.

In certain embodiments, a subject is administered a superantigen conjugate, e.g., a superantigen conjugate contemplated herein, daily for 2 to 6 consecutive days (e.g., 2, 3, 4, 5, or 6 consecutive days) every 2 to 12 weeks (e.g., 2, 3, 4, 5, 6, 7, 8, 9, 10, 11, or 12 weeks). In certain embodiments, a subject is administered a superantigen conjugate, e.g., a superantigen conjugate contemplated herein, daily for 4 consecutive days every 3 to 4 weeks (e.g., 3 or 4 weeks). In certain embodiments, a subject is administered a superantigen conjugate, e.g., a superantigen conjugate contemplated herein, once every 2 to 8 weeks (e.g., once every 2, 3, 4, 5, 6, 7, or 8 weeks).

The methods and compositions described herein can be used alone or in combination with other therapeutic agents and/or modalities. The term administered “in combination,” as used herein, is understood to mean that two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In certain embodiments, the treatment regimen of the present invention may involve contacting the neoplasm or tumor cells with a first treatment and a second treatment at the same time. This may be achieved by contacting the cell with a single composition or pharmacological formulation that includes both agents, or by contacting the cell with two distinct compositions or formulations, at the same time, wherein one composition includes the first treatment and the other includes the second treatment.

Alternatively, the first treatment may precede or follow the second treatment by intervals ranging from minutes, days to weeks. In embodiments where the first treatment and the second treatment are applied separately to the cell, one should ensure that a significant period of time does not expire between the time of each delivery, such that the first treatment and the second treatment would still be able to exert an advantageously combined effect on the cell. In such instances, it is contemplated that one may contact the cell with both modalities within about 12-72 hours of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

Various combinations may be employed, the first treatment being “A” and the second treatment being “B”: A/B/A, B/A/B, B/B/A, A/A/B, A/B/B, B/A/A, A/B/B/B, B/A/B/B, B/B/B/A, B/B/A/B, A/A/B/B, A/B/A/B, A/B/B/A, B/B/A/A, B/A/B/A, B/A/A/B, A/A/A/B, B/A/A/A, A/B/A/A, and A/A/B/A.

In certain embodiments, a method or composition described herein, is administered in combination with one or more additional therapies, e.g., surgery, radiation therapy, or administration of another therapeutic preparation. In certain embodiments, the additional therapy may include chemotherapy, e.g., a cytotoxic agent. In certain embodiments the additional therapy may include a targeted therapy, e.g. a tyrosine kinase inhibitor, a proteasome inhibitor, or a protease inhibitor. In certain embodiments, the additional therapy may include an anti-inflammatory, anti-angiogenic, anti-fibrotic, or anti-proliferative compound, e.g., a steroid, a biologic immunomodulator, a monoclonal antibody, an antibody fragment, an aptamer, an siRNA, an antisense molecule, a fusion protein, a cytokine, a cytokine receptor, a bronchodialator, a statin, an anti-inflammatory agent (e.g. methotrexate), or an NSAID. In certain embodiments, the additional therapy may include a compound designed to reduce the subject's possible immunoreactivity to the administered 5T4-targeting agent. For example, immunoreactivity to the administered 5T4-targeting agent may be reduced via co-administration with, for example, an anti-CD20 antibody and/or an anti-CD19 antibody, that reduces the production of anti-drug antibodies in the subject. In certain embodiments, the additional therapy may include a combination of therapeutics of different classes.

In certain embodiments, a method or composition described herein is administered in combination with an immunopotentiator.

In certain embodiments, exemplary immunopotentiators can: (a) stimulate activating T-cell signaling, (b) repress T-cell inhibitory signalling between the cancerous cells and a T-cell, (c) repress inhibitory signalling that leads to T-cell expansion, activation and/or activity via a non-human IgG1-mediated immune response pathway, for example, a human IgG4 immunoglobulin-mediated pathway, (d) a combination of (a) and (b), (e) combination of (a) and (c), (f) a combination of (b) and (c), and (g) a combination of (a), (b), and (c).

In certain embodiments, the immunopotentiator is a checkpoint pathway inhibitor. The checkpoint inhibitor may, for example, be selected from a PD-1 antagonist, PD-L1 antagonist, CTLA-4 antagonist, adenosine A2A receptor antagonist, B7-H3 antagonist, B7-H4 antagonist, BTLA antagonist, KIR antagonist, LAG3 antagonist, TIM-3 antagonist, VISTA antagonist or TIGIT antagonist.

PD-1 is a receptor present on the surface of T-cells that serves as an immune system checkpoint that inhibits or otherwise modulates T-cell activity at the appropriate time to prevent an overactive immune response. Cancer cells, however, can take advantage of this checkpoint by expressing ligands, for example, PD-L1, PD-L2, etc., that interact with PD-1 on the surface of T-cells to shut down or modulate T-cell activity. Using this approach, cancer can evade the T-cell mediated immune response.

In the CTLA-4 pathway, the interaction of CTLA-4 on the T-cell with its ligands (e.g., CD80, also known as B7-1, and CD86) on the surface of an antigen presenting cells (rather than the cancer calls) leads to T-cell inhibition. As a result, the ligand that inhibits T-cell activation or activity (e.g., CD80 or CD86) is provided by an antigen presenting cell (a key cell type in the immune system) rather than the cancer cell. Although CTLA-4 and PD-1 binding both have similar negative effects on T-cells the timing of down regulation, the responsible signaling mechanisms, and the anatomic locations of immune inhibition by these two immune checkpoints differ (Buchbinder et al. (2016) AMERICAN JOURNAL OF CLINICAL ONCOLOGY, 39(1)). Unlike CTLA-4, which is confined to the early priming phase of T-cell activation, PD-1 functions much later during the effector phase, (Keir et al. (2008) ANNU. REV IMMUNOL., 26:677-704). CTLA-4 and PD-1 represent two T-cell-inhibitory receptors with independent, non-redundant mechanisms of action.

In certain embodiments, the immunopotentiator prevents (completely or partially) an antigen expressed by the cancerous cell from repressing T-cell inhibitory signaling between the cancerous cell and the T-cell. In one embodiment, such an immunopotentiator is a checkpoint inhibitor, for example, a PD-1-based inhibitor. Examples of such immunopotentiators include, for example, anti-PD-1 antibodies, anti-PD-L1 antibodies, and anti-PD-L2 antibodies.

In certain embodiments, the 5T4-targeting agent is administered with a PD-1-based inhibitor. A PD-1-based inhibitor can include (i) a PD-1 inhibitor, i.e., a molecule (for example, an antibody or small molecule) that binds to PD-1 on a T-cell to prevent the binding of a PD-1 ligand expressed by the cancer cell of interest, and/or (ii) a PD-L inhibitor, e.g., a PD-L1 or PD-L2 inhibitor, i.e., a molecule (for example, an antibody or small molecule) that binds to a PD-1 ligand (for example, PD-L1 or PD-L2) to prevent the PD-1 ligand from binding to its cognate PD-1 on the T-cell.

In certain embodiments the 5T4-targeting agent is administered with a CTLA-4 inhibitor, e.g., an anti-CTLA-4 antibody. Exemplary anti-CTLA-4 antibodies are described in U.S. Pat. Nos. 6,984,720, 6,682,736, 7,311,910; 7,307,064, 7,109,003, 7,132,281, 6,207,156, 7,807,797, 7,824,679, 8,143,379, 8,263,073, 8,318,916, 8,017,114, 8,784,815, and 8,883,984, International (PCT) Publication Nos. WO98/42752, WO00/37504, and WO01/14424, and European Patent No. EP 1212422 B1. Exemplary CTLA-4 antibodies include ipilimumab or tremelimumab.

In certain embodiments, the immunopotentiator prevents (completely or partially) an antigen expressed by the cancerous cell from repressing T-cell expansion, activation and/or activity via a human IgG4 (a non-human IgG1) mediated immune response pathway, for example, not via an ADCC pathway. It is contemplated that, in some embodiments, although the immune response potentiated by the immunopotentiator may include some ADCC activity, the principal component(s) of the immune response do not involve ADCC activity. In contrast, some of the antibodies currently being used in immunotherapy, such as ipilimumab (an anti-CTLA-4 IgG1 monoclonal antibody), can kill targeted cells via ADCC through signaling via their Fc domain through Fc receptors on effector cells. Ipilimumab, like many other therapeutic antibodies, was designed as a human IgG1 immunoglobulin, and although ipilimumab blocks interactions between CTLA-4 and CD80 or CD86, its mechanism of action is believed to include ADCC depletion of tumor-infiltrating regulatory T-cells that express high levels of cell surface CTLA-4. (Mahoney et al. (2015) NATURE REVIEWS, DRUG DISCOVERY 14: 561-584.) Given that CTLA-4 is highly expressed on a subset of T-cells (regulatory T-cells) that act to negatively control T-cells activation, when an anti-CTLA-4 IgG1 antibody is administered, the number of regulatory T-cells is reduced via ADCC.

In certain embodiments, it is desirable to use immunopotentiators whose mode of action is primarily to block the inhibitory signals between the cancer cells and the T-cells without significantly depleting the T-cell populations (for example, permitting the T-cell populations to expand). To achieve this, it is desirable to use an antibody, for example, an anti-PD-1 antibody, an anti-PD-L1 antibody or an anti-PD-L2 antibody, that has or is based on a human IgG4 isotype. Human IgG4 isotype is preferred under certain circumstances because this antibody isotype invokes little or no ADCC activity compared to the human IgG1 isotype (Mahoney et al. (2015) supra). Accordingly, in certain embodiments, the immunopotentiator, e.g., the anti-PD-1 antibody, anti-PD-L1 antibody, or anti-PD-L2 antibody has or is based on a human IgG4 isotype. In certain embodiments, the immunopotentiator is an antibody not known to deplete Tregs, e.g., IgG4 antibodies directed at non-CTLA-4 checkpoints (for example, anti-PD-1 IgG4 inhibitors).

In certain embodiments, the immunopotentiator is an antibody that has or is based on a human IgG1 isotype or another isotype that elicits antibody-dependent cell-mediated cytotoxicity (ADCC) and/or complement mediated cytotoxicity (CDC). In other embodiments, the immunopotentiator is an antibody that has or is based on a human IgG4 isotype or another isotype that elicits little or no antibody-dependent cell-mediated cytotoxicity (ADCC) and/or complement mediated cytotoxicity (CDC).

Exemplary PD-1-based inhibitors are described in U.S. Pat. Nos. 8,728,474, 8,952,136, and 9,073,994, and EP Patent No. 1537878B1. Exemplary anti-PD-1 antibodies are described, for example, in U.S. Pat. Nos. 8,952,136, 8,779,105, 8,008,449, 8,741,295, 9,205,148, 9,181,342, 9,102,728, 9,102,727, 8,952,136, 8,927,697, 8,900,587, 8,735,553, and 7,488,802. Exemplary anti-PD-1 antibodies include nivolumab (OPDIVO®, Bristol-Myers Squibb), pembrolizumab (KEYTRUDA®, Merck), cemiplimab (LIBTAYO®, Regeneron/Sanofi), spartalizumab (PDR001), MED10680 (AMP-514), pidilizumab (CT-011), dostarlimab (TSR-042, WBP-285), sintilimab (IBI308), toripalimab (JS 001), camrelizumab (SHR1210), pimivalimab (JTX-4014), tislelizumab (BGB-A317), prolgolimab, INCMGA00012 (MGA012), and AMP-514. Exemplary anti-PD-L1 antibodies are described, for example, in U.S. Pat. Nos. 9,273,135, 7,943,743, 9,175,082, 8,741,295, 8,552,154, and 8,217,149. Exemplary anti-PD-L1 antibodies include avelumab (BAVENCIO®, EMD Serono/Pfizer), atezolizumab (TECENTRIQ®, Genentech), and durvalumab (IMFINZI®, Medimmune/AstraZeneca), CS1001 (CStone Therapeutics), tagitanlimab (KL-A167), cosibelimab (CK-301; Checkpoint Therapeutics), TQB2450, envafolimab (KN035), SHR-1316, STI-A1014, BGB-A333, MSB2311, HLX-20, and BMS-936559 (Bristol-Myers Squibb).

In certain embodiments, a subject is administered a PD-1-based inhibitor, e.g., an anti-PD-1 antibody, e.g., an anti-PD-1 antibody contemplated herein, every 1 to 5 weeks (e.g., every 1, 2, 3, 4, or 5 weeks). In certain embodiments, a subject is administered a PD-1-based inhibitor, e.g., an anti-PD-1 antibody, e.g., an anti-PD-1 antibody contemplated herein, every 2 to 4 weeks (e.g., every 2, 3, or 4 weeks).

The PD-1-based inhibitor may be designed, expressed, and purified using techniques known to those skilled in the art, for example, as described hereinabove. The anti-PD-1 antibodies may be designed, expressed, purified, formulated and administered as described in U.S. Pat. Nos. 8,728,474, 8,952,136, and 9,073,994.

Other immunopotentiators (for example, antibodies, and various small molecules) may target signaling pathways involving one or more of the following ligands. B7-H3 (found on prostrate, renal cell, non-small cell lung, pancreatic, gastric, ovarian, colorectal cells, among others); B7-H4 (found on breast, renal cell, ovarian, pancreatic, melanoma cells, among others); HHLA2 (found on breast, lung, thyroid, melanoma, pancreas, ovary, liver, bladder, colon, prostate, kidney cells, among others); galectins (found on non-small cell lung, colorectal, and gastric cells, among others); CD30 (found on Hodgkin lymphoma, large cell lymphoma cells, among others): CD70 (found on non-Hodgkin's lymphoma, renal cells, among others); ICOSL (found on glioblastoma, melanoma cells, among others); CD155 (found on kidney, prostrate, pancreatic glioblastoma cells, among others); and TIM3. Similarly, other potential immunopotentiators that can be used include, for example, a 4-1BB (CD137) agonist (e.g., the fully human IgG4 anti-CD137 antibody Urelumab/BMS-663513), a LAG3 inhibitor (e.g., the humanized IgG4 anti-LAG3 antibody LAG525, Novartis): an IDO inhibitor (e.g., the small molecule INCB024360, Incyte Corporation), a TGFβ inhibitor (e.g., the small molecule Galunisertib, Eli Lilly) and other receptor or ligands that are found on T-cells and/or tumor cells. In certain embodiments, immunopotentiators (for example, antibodies, and various small molecules) that target signaling pathways involving one or more of the foregoing ligands are amenable to pharmaceutical intervention based on agonist/antagonist interactions but not through ADCC.

It is further envisioned that the present invention can be used in combination with surgical intervention. In the case of surgical intervention, the present invention may be used preoperatively, e.g., to render an inoperable tumor subject to resection. Alternatively, the present invention may be used at the time of surgery, and/or thereafter, to treat residual or metastatic disease. For example, a resected tumor bed may be injected or perfused with a formulation comprising the 5T4-targeting agent. The perfusion may be continued post-resection, for example, by leaving a catheter implanted at the site of the surgery. Periodic post-surgical treatment also is envisioned. Any combination of the invention therapy with surgery is within the scope of the invention.

Continuous administration also may be applied where appropriate, for example, where a tumor is excised and the tumor bed is treated to eliminate residual, microscopic disease. Delivery via syringe or cauterization is preferred. Such continuous perfusion may take place for a period from about 1-2 hours, to about 2-6 hours, to about 6-12 hours, to about 12-24 hours, to about 1-2 days, to about 1-2 weeks or longer following the initiation of treatment. Generally, the dose of the therapeutic composition via continuous perfusion will be equivalent to that given by a single or multiple injections, adjusted over a period of time during which the perfusion occurs. It is further contemplated that limb perfusion may be used to administer therapeutic compositions of the present invention, particularly in the treatment of melanomas and sarcomas.

Exemplary cytotoxic agents that can be administered in combination with a method or composition described herein include, for example, antimicrotubule agents, topoisomerase inhibitors, antimetabolites, protein synthesis and degradation inhibitors, mitotic inhibitors, alkylating agents, platinating agents, inhibitors of nucleic acid synthesis, histone deacetylase inhibitors (HDAC inhibitors, e.g., vorinostat (SAHA, MK0683), entinostat (MS-275), panobinostat (LBH589), trichostatin A (TSA), mocetinostat (MGCD0103), belinostat (PXD101), romidepsin (FK228, depsipeptide)), DNA methyltransferase inhibitors, nitrogen mustards, nitrosoureas, ethylenimines, alkyl sulfonates, triazenes, folate analogs, nucleoside analogs, ribonucleotide reductase inhibitors, vinca alkaloids, taxanes, epothilones, intercalating agents, agents capable of interfering with a signal transduction pathway, agents that promote apoptosis and radiation, or antibody molecule conjugates that bind surface proteins to deliver a toxic agent. In one embodiment, the cytotoxic agent that can be administered with a method or composition described herein is a platinum-based agent (such as cisplatin), cyclophosphamide, dacarbazine, methotrexate, fluorouracil, gemcitabine, capecitabine, hydroxyurea, topotecan, irinotecan, azacytidine, vorinostat, ixabepilone, bortezomib, taxanes (e.g., paclitaxel or docetaxel), cytochalasin B, gramicidin D, ethidium bromide, emetine, mitomycin, etoposide, tenoposide, vincristine, vinblastine, vinorelbine, colchicin, anthracyclines (e.g., doxorubicin or epirubicin) daunorubicin, dihydroxy anthracin dione, mitoxantrone, mithramycin, actinomycin D, adriamycin, 1-dehydrotestosterone, glucocorticoids, procaine, tetracaine, lidocaine, propranolol, puromycin, ricin, or maytansinoids.

### EXAMPLES

The following Examples are merely illustrative and are not intended to limit the scope or content of the invention in any way.

### Example 1

This Example describes an expression analysis of 5T4 in human glioblastoma (GBM) tumor tissues.

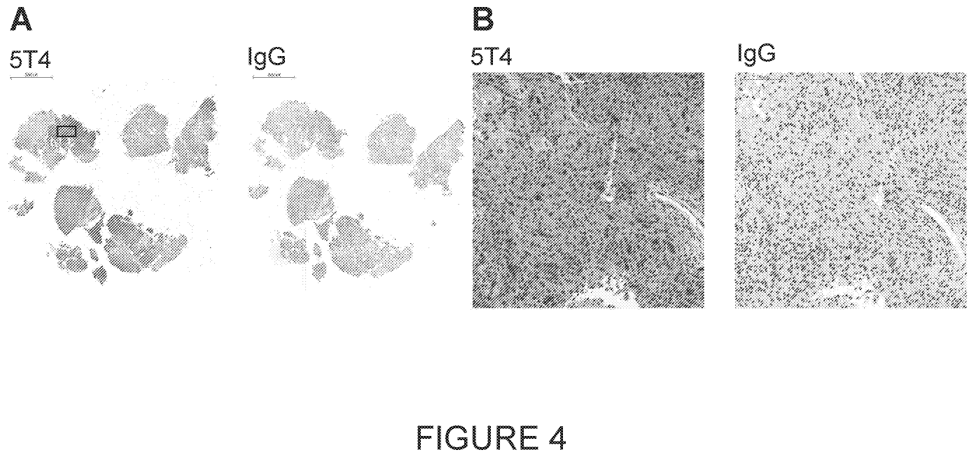

Human GBM tissue samples were obtained from a commercial source and analyzed by immunohistochemistry (IHC) using a rabbit anti-human 5T4 antibody (clone EPR5529; Abcam, ab134162). The stained samples were scored for 5T4 prevalence by a trained pathologist. Prevalence was defined as the percentage of tumor cores having at least 0.5% tumor cells with membrane 5T4 expression. 30 samples were analyzed. Unexpectedly, 87% (26/30) of the analyzed GBM tissue samples demonstrated tumor membrane positivity. Of the 26 positive cases, 67% (21/30) had up to 3% of tumor cells with membrane 5T4 expression and 17% (5/30) had more than 10% of tumor cells with membrane 5T4 expression. Representative IHC images are shown in FIG. 4.

### Example 2

This Example describes an analysis of 5T4 membrane expression in the human glioblastoma (GBM) cell lines U-138, U-87, and GMS-10. A human renal cell carcinoma (RCC) cell line known to express high levels of 5T4 (Caki-2) and a human ovarian cancer cell line known to express low levels of 5T4 (OVCAR-3) were used as controls. 5T4 membranal expression was determined by flow cytometry.

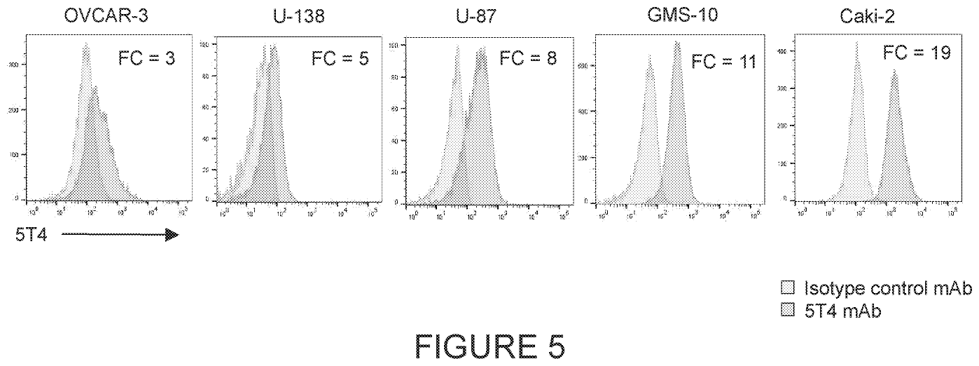

The cell lines were analyzed using flow cytometry with an Alexa Fluor 647-conjugated mouse IgG1 anti-human 5T4 monoclonal antibody (clone 524744; R&D systems, FAB49751R). Alexa Fluor 647-conjugated mouse IgG1 isotype control antibody was used as a negative control for staining. The cell lines were washed with PBS, labeled with viability dye (Zombie Violet, BioLegend 77477 or Zombie NIR, Biolegend 423105), and then incubated with the indicated mAb in flow cytometry buffer (PBS, 0.5% BSA, 2 mM EDTA, 0.05% NaN3) for 30 minutes at room temperature. Cells were subsequently washed with flow cytometry buffer and acquired on a MACSQuant flow cytometer (Miltenyi Biotec). Dead cells were gated out of the analysis. Data analysis was performed using FlowJo 10 software. As seen in FIG. 5, all three GBM cell lines U-138, U-87, and GMS-10 were positive for 5T4 expression. The fold change (FC) of 5T4 mAb staining over isotype control for the U-138, U-87, and GMS-10 cell lines was 5, 8, and 11, respectively, which was higher than the expression on the OVCAR-3 cell line (FC=3) but lower than the expression on the Caki-2 cell line (FC=19).

### Example 3

This example describes an in vitro study testing the anti-cancer effect of T cells in combination with the 5T4-targeted superantigen naptumomab estafenatox (NAP; ANYARA®) against the human glioblastoma (GBM) cell lines U-138, U-87, and GMS-10.

Peripheral blood mononuclear cells (PBMCs) were isolated from healthy donors. PBMCs were incubated for 4 days with 10 ng/ml of Staphylococcal enterotoxin A (SEA). T cells were then isolated and incubated with IL-2 for 1 additional day. 8×10³ U-138, U-87, or GMS-10 cells per well were incubated in 96-well plates for 1 hour with T cells. The effector:target ratio (T cells:GBM cells) was 10:1. After 1 hour of incubation, NAP at different concentrations (ranging from 0 to 10 μg/ml) was added to the wells and the plates were incubated for an additional 4-48 hours. At the end of the treatment the culture supernatant was removed, including suspended T cells and tumor cells, and the attached tumor cells were washed once with culture medium. The viability of residual GBM cells was tested with a CCK8 kit (Cell Counting Kit-8, Sigma Aldrich) according to the manufacturer's protocol. The viability of the control group was normalized to 100%. Viability of the cancer cells (%)=(OD value of treatment group/OD value of the control group)×100. Data were analyzed using Graph Pad Prism software.

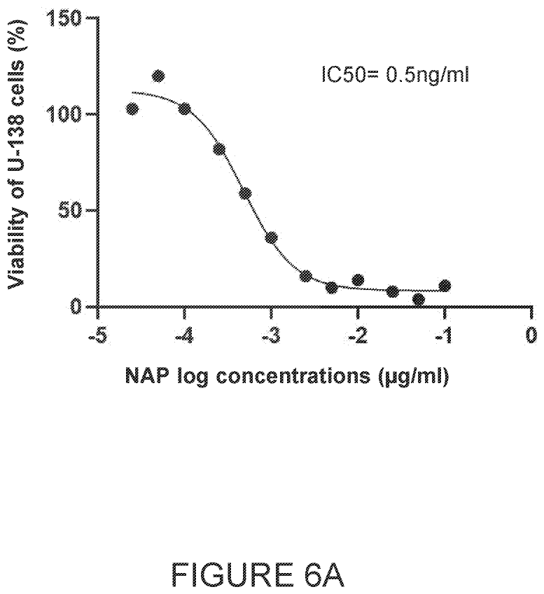

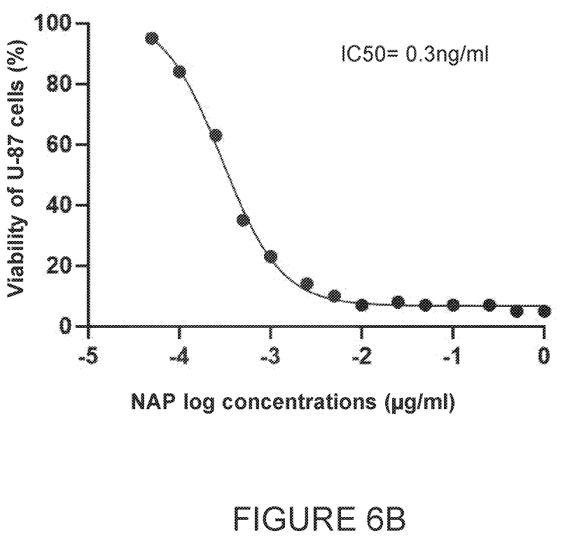

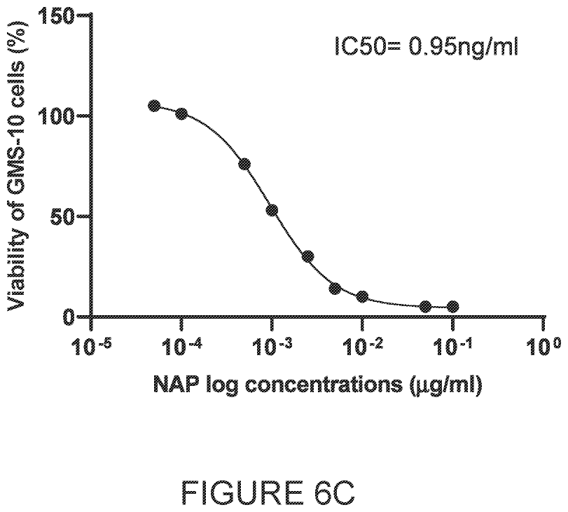

Dose-response curves are shown for three GBM cell lines: U-138 (FIG. 6A), U-87 (FIG. 63), and GMS10 (FIG. 6C). The combination of T cells and NAP reduced viability of the U-138, U-87, and GMS-10 cancer cells, and the IC50 of NAP was 0.5 ng/ml, 0.3 ng/ml, and 0.95 ng/ml, respectively (4-13 pM). These data demonstrate that NAP is highly potent against all three 5T4-expressing GBM cell-lines, including the GBM cell lines with a relatively low level of expression of 5T4.

### Example 4

This example describes an in vitro study testing whether the anti-cancer effect of T cells in combination with the 5T4-targeted superantigen NAP against the GMS-10 human glioblastoma (GBM) cell line is mediated by the 5T4 antigen.

PBMCs were isolated from healthy donors. PBMCs were incubated for 4 days with 10 ng/ml of Staphylococcal enterotoxin A (SEA). T cells were then isolated and incubated with IL-2 for 1 additional day. 2×10⁴ carboxyfluorescein succinimidyl ester (CFSE)-labeled GMS-10 cells per well were incubated in 96-well plates for 24 hours. These cells were incubated with 0-50 μg/ml of an anti-5T4 Fab, 5T4FabV18, in order to block the 5T4 antigen. Following the 24-hour incubation, the GMS-10 cells were washed and the pre-activated T cells were added to the wells at an effector:target (T cells:GMS-10 cells) ratio of 10:1. After 1 hour of incubation, NAP in different concentrations (0-0.005 μg/ml) was added to the wells and the plates were incubated for an additional 48 hours. At the end of the treatment the culture supernatant was removed, including suspended T cells and tumor cells. The attached tumor cells were detached, washed once with PBS, and stained for dead cells. The viability of residual GMS-10 cells was determined by FACS. The viability of CFSE gated cells in the control group was normalized to 100%. Viability of the cancer cells (%)=(cell count value of treatment group/cell count value of control group)×100.

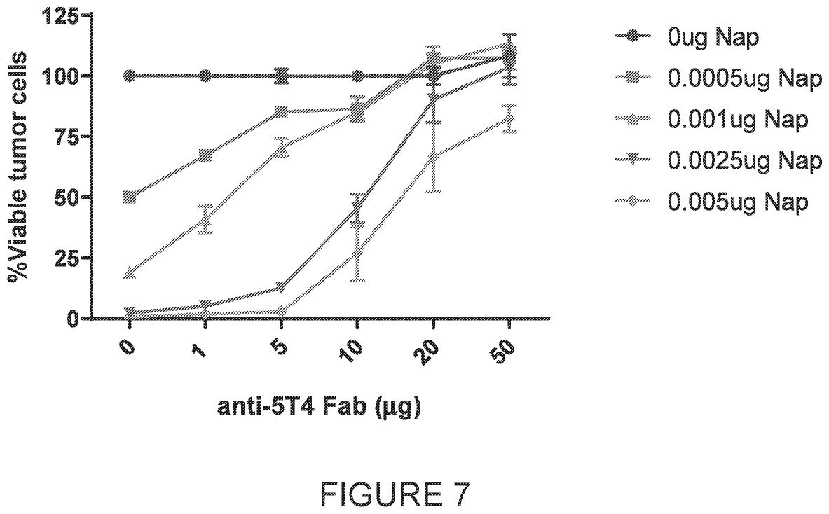

The results of this study are presented in FIG. 7. As depicted, blocking the 5T4 antigen with an anti-5T4 Fab diminished the anti-cancer effect of NAP in a dose-dependent manner. These data demonstrate that NAP activity against the GMS-10 GBM cell line is mediated by the 5T4 antigen expressed on the surface of GMS-10 cells.

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

SEQUENCE LISTING

| SEQ | |
|---|---|
| ID | |
| NO: | Sequence |
| 1 | SEKSEEINEKDLRKKSELQRNALSNLRQIYYYNEKAITENKESDDQFLENTLLFKGFFTG |
| | HPWYNDLLVDLGSKDATNKYKGKKVDLYGAYYGYQCAGGTPNKTACMYGGVTLHDNNRLT |
| | EEKKVPINLWIDGKQTTVPIDKVKTSKKEVTVQELDLQARHYLHGKFGLYNSDSFGGKVQ |
| | RGLIVFHSSEGSTVSYDLFDAQGQYPDTLLRIYRDNKTINSENLHIDLYLYTT |
| 2 | SEKSEEINEKDIRKKSELQGTALGNLKQIYYYNEKAKTENKESHDQFLQHTILFKGFFTD |
| | HSWYNDLLVDFDSKDIVDKYKGKKVDLYGAYYGYQCAGGTPNKTACMYGGVTLHDNNRLT |
| | EEKKVPINLWLDGKQNTVPLETVKTNKKNVTVQELDLQARRYLQEKYNLYNSDVEDGKVQ |
| | RGLIVFHTSTEPSVNYDLFGAQGQYSNTLLRIYRDNKTINSENMHIDIYLYTS |
| 3 | SEKSEEINEKDIRKKSELQGTALGNLKQIYYYNSKAITSSEKSADQFLTNTLLFKGFFTG |
| | HPWYNDLLVDLGSTAATSEYEGSSVDLYGAYYGYQCAGGTPNKTACMYGGVTLHDNNRLT |
| | EEKKVPINLWIDGKQTTVPIDKVKTSKKEVTVQELDLQARHYLHGKFGLYNSDSFGGKVQ |
| | RGLIVFHSSEGSTVSYDLFDAQGQYPDTLLRIYRDNTTISSTSLSISLYLYTT |
| 4 | SEKSEEINEKDLRKKSELQGTALGNLKQIYYYNEKAKTENKESHDQFLQHTILFKGFFTD |
| | HSWYNDLLVDFDSKDIVDKYKGKKVDLYGAYYGYQCAGGTPNKTACMYGGVTLHDNNRLT |
| | EEKKVPINLWLDGKQNTVPLETVKTNKKNVTVQELDLQARRYLQEKYNLYNSDVFDGKVQ |
| | RGLIVFHTSTEPSVNYDLFGAQGQYSNTLLRIYRDNKTINSENMHIAIYLYTS |
| 5 | QVQLQQPGAELVRPGASVKLSCKASGYTFTNYWINWVKQRPGQGLEWIGNIYPSYIYTNY |
| | NQEFKDKVTLTVDESSSTAYMQLSSPTSEDSAVYYCTRSPYGYDEYGLDYWGQGTSVTVS |
| | SAKTTPPSVYPLAPGSAAQTNSMVTLGCLVKGYFPEPVTVTWNSGSLSSGVHTFPAVLQS |
| | DLYTLSSSVTVPSSTWPSETVTCNVAHPASSTKVDKKIVPRDSGGPSEKSEEINEKDLRK |
| | KSELQGTALGNLKQIYYYNEKAKTENKESHDQFLQHTILFKGFFTDHSWYNDLLVDFDSK |
| | DIVDKYKGKKVDLYGAYYGYQCAGGTPNKTACMYGGVTLHDNNRLTEEKKVPINLWLDGK |
| | QNTVPLETVKTNKKNVTVQELDLQARRYLQEKYNLYNSDVFDGKVQRGLIVFHTSTEPSV |
| | NYDLFGAQGQYSNTLLRIYRDNKTINSENMHIDIYLYTSDIVMTQSPSSLTVTAGEKVTM |
| | NCKSSQSLINSRNQKNYLTWYQQKPGQPPKLLIYWASTRESGVPDRFTGSGSGTDFTITI |
| | SSVQAEDLAVYYCQNDYVYPLTFGAGTKLELKRADAAPTVSIFPPSSEQLTSGGASVVCF |
| | LNNFYPKDINVKWKIDGSERQNGVLNSWTDQDSKDSTYSMSSTLTLTKDEYERHNSYTCE |
| | ATHKTSTSPIVKSFNRNES |
| 6 | EVQLQQSGPDLVKPGASVKISCKASGYSFTGYYMHWVKQSHGKSLEWIGRINPNNGVTLY |
| | NQKFKDKAILTVDKSSTTAYMELRSLTSEDSAVYYCARSTMITNYVMDYWGQVTSVTVSS |
| | AKTTPPSVYPLAPGSAAQTNSMVTLGCLVKGYFPEPVTVTWNSGSLSSGVHTFPAVLQSD |
| | LYTLSSSVTVPSSTWPSETVTCNVAHPASSTKVDKKIVPRDSGGPSEKSEEINEKDLRKK |
| | SELQGTALGNLKQIYYYNEKAKTENKESHDQFLQHTILFKGFFTDHSWYNDLLVDFDSKD |
| | IVDKYKGKKVDLYGAYYGYQCAGGTPNKTACMYGGVTLHDNNRLTEEKKVPINIWLDGKQ |
| | NTVPLETVKTNKKNVTVQELDLQARRYLQEKYNLYNSDVEDGKVQRGLIVFHTSTEPSVN |
| | YDLFGAQGQYSNTLLRIYRDNKTINSENMHIAIYLYTSSIVMTQTPTSLLVSAGDRVTIT |
| | CKASQSVSNDVAWYQQKPGQSPKLLISYTSSRYAGVPDRFSGSGSGTDFTLTISSVQAED |
| | LAVYFCQQDYNSPPTFGGGTKLEIKRADAAPTVSIFPPSSEQLTSGGASVVCFLNNFYPK |
| | DINVKWKIDGSERQNGVLNSWTDQDSKDSTYSMSSTLTLTKDEYERHNSYTCEATHKTST |
| | SPIVKSFNRNES |
| 7 | EVQLQQSGPDLVKPGASVKISCKASGYSFTGYYMHWVKQSPGKGLEWIGRINPNNGVTLY |
| | NQKFKDKATLTVDKSSTTAYMELRSLTSEDSAVYYCARSTMITNYVMDYWGQGTSVTVSS |
| | AKTTPPSVYPLAPGSAAQTNSMVTLGCLVKGYFPEPVTVTWNSGSLSSGVHTFPAVLQSD |
| | LYTLSSSVTVPSSTWPSETVTCNVAHPASSTKVDKKIVPRDSGGPSEKSEEINEKDLRKK |
| | SELQGTALGNLKQIYYYNSKAITSSEKSADQFLTNTLLFKGFFTGHPWYNDLLVDLGSTA |
| | ATSEYEGSSVDLYGAYYGYQCAGGTPNKTACMYGGVTLHDNNRLTEEKKVPINLWIDGKQ |
| | TTVPIDKVKTSKKEVTVQELDLQARHYLHGKFGLYNSDSEGGKVQRGLIVFHSSEGSTVS |
| | YDLFDAQGQYPDTLLRIYRDNTTISSTSLSISLYLYTTSIVMTQTPTSLLVSAGDRVTIT |
| | CKASQSVSNDVAWYQQKPGQSPKLLISYTSSRYAGVPDRFSGSGYGTDFTITISSVQAED |
| | AAVYFCQQDYNSPPTFGGGTKLEIKRADAAPTVSIFPPSSEQLTSGGASVVCFLNNFYPK |
| | DINVKWKIDGSERQNGVLNSWTDQDSKDSTYSMSSTLTLTKDEYERHNSYTCEATHKTST |
| | SPIVKSFNRNES |
| 8 | EVQLQQSGPDLVKPGASVKISCKASGYSFTGYYMHWVKQSPGKGLEWIGRINPNNGVTLY |
| | NQKFKDKATLTVDKSSTTAYMELRSLTSEDSAVYYCARSTMITNYVMDYWGQGTSVTVSS |
| | AKTTPPSVYPLAPGSAAQTNSMVTLGCLVKGYFPEPVTVTWNSGSLSSGVHTFPAVLQSD |
| | LYTLSSSVTVPSSTWPSETVTCNVAHPASSTKVDKKIVPRDSGGPSEKSEEINEKDLRKK |
| | SELQGTALGNLKQIYYYNSKAITSSEKSADQFLTNTLLFKGFFTGHPWYNDLLVDLGSTA |
| | ATSEYEGSSVDLYGAYYGYQCAGGTPNKTACMYGGVTLHDNNRLTEEKKVPINLWIDGKQ |
| | TTVPIDKVKTSKKEVTVQELDLQARHYLHGKFGLYNSDSFGGKVQRGLIVFHSSEGSTVS |
| | YDLFDAQGQYPDTLLRIYRDNTTISSTSLSISLYLYTT |
| 9 | SIVMTQTPTSLLVSAGDRVTITCKASQSVSNDVAWYQQKPGQSPKLLISYTSSRYAGVPD |
| | RFSGSGYGTDFTLTISSVQAEDAAVYFCQQDYNSPPTFGGGTKLEIKRADAAPTVSIFPP |
| | SSEQLTSGGASVVCFLNNFYPKDINVKWKIDGSERQNGVLNSWTDQDSKDSTYSMSSTLT |
| | LTKDEYERHNSYTCEATHKTSTSPIVKSFNRNES |
| 10 | SEKSEEINEKDLRKKSELQGTALGNLKQIYYYNEKAITENKESDDQFLENTLLEKGFFTG |
| | HPWYNDLLVDLGSKDATNKYKGKKVDLYGAYYGYQCAGGTPNKTACMYGGVTLHDNNRLT |
| | EEKKVPINLWIDGKQTTVPIDKVKTSKKEVTVQELDLQARHYLHGKFGLYNSDSFGGKVQ |
| | RGLIVFHSSEGSTVSYDLFDAQGQYPDTLLRIYRDNKTINSENLHIALYLYTT |

What is claimed is:

1. A method of reducing tumor volume in a subject with glioblastoma, the method comprising administering to the subject an effective amount of a 5T4-targeting agent.

2. A method of killing tumor cells in a subject with glioblastoma, the method comprising administering to the subject an effective amount of a 5T4-targeting agent.

3. A method of treating glioblastoma in a subject in need thereof, the method comprising administering to the subject an effective amount of a 5T4-targeting agent.

4. The method of any one of claims 1-3, wherein at least 0.5%, 1%, 2%, 3%, 4%, 5%, 10%, 15%, 20%, 25%, or 30% of (i) tumor cells in the subject, (ii) tumor cells in a tumor in the subject, and/or (iii) tumor cells in a tumor sample (e.g., a tumor tissue sample) from the subject, exhibit cell membrane expression of 5T4, as measured by immunohistochemistry.

5. The method of any one of claims 1-4, wherein 0.5% to 10% (e.g., 0.5% to 3%, 3% to 5%, or 5% to 10%) of (i) tumor cells in the subject, (ii) tumor cells in a tumor in the subject, and/or (iii) tumor cells in a tumor sample (e.g., a tumor tissue sample) from the subject, exhibit cell membrane expression of 5T4, as measured by immunohistochemistry.

6. The method of any one of claims 1-5, wherein the 5T4-targeting agent comprises an antibody, a bispecific T-cell engager (BiTE), an immune cell, or a vaccine.

7. The method of any one of claims 1-6, wherein the 5T4-targeting agent is a superantigen conjugate comprising a superantigen covalently linked to an anti-5T4 antibody.

8. The method of claim 7, wherein the superantigen comprises Staphylococcal enterotoxin A or an immunologically variant and/or fragment thereof.

9. The method of claim 7 or 8, wherein the superantigen comprises the amino acid sequence of SEQ ID NO: 3, or an immunologically reactive variant and/or fragment thereof.

10. The method of any one of claims 7-9, wherein the anti-5T4 antibody comprises a Fab fragment that binds a 5T4 cancer antigen.

11. The method of any one of claims 7-10, wherein the anti-5T4 antibody comprises a heavy chain comprising amino acid residues 1-222 of SEQ ID NO: 8 and a light chain comprising amino acid residues 1-214 of SEQ ID NO: 9.

12. The method of any one of claims 7-11, wherein the superantigen conjugate comprises a first protein chain comprising SEQ ID NO: 8 and a second protein chain comprising SEQ ID NO: 9.

13. The method of claim 6, wherein the 5T4-targeting agent is an immune cell.

14. The method of claim 13, wherein the immune cell is a chimeric antigen receptor (CAR)-expressing immune cell.

15. The method of claim 13 or 14, wherein the immune cell is a T-cell or a Natural Killer (NK) cell.

16. The method of claim 15, wherein the immune cell is a T-cell.

17. The method of any one of claims 1-16, wherein the method further comprises administering to the subject an immunopotentiator.

18. The method of claim 17, wherein the immunopotentiator is a CTLA-4- or a PD-1-based inhibitor.

19. The method of claim 18, wherein the PD-1-based inhibitor is a PD-1 or PD-L1 inhibitor.

20. The method of claim 19, wherein the PD-1 inhibitor is an anti-PD-1 antibody.

21. The method of claim 20, wherein the anti-PD-1 antibody is selected from nivolumab pembrolizumab, cemiplimab, spartalizumab, MEDI0680, pidilizumab, dostarlimab, sintilimab, toripalimab, camrelizumab, pimivalimab, tislelizumab, and prolgolimab.

22. The method of claim 19, wherein the PD-L1 inhibitor is an anti-PD-L1 antibody.

23. The method of claim 22, wherein the anti-PD-L1 antibody is selected from atezolizumab, avelumab, durvalumab, CS1001, tagitanlimab, cosibelimab, TQB2450, envafolimab, SHR-1316, STI-A1014, BGB-A333, MSB2311, HLX-20, and BMS-936559.

24. The method of any one of claims 1-23, wherein the subject is a human subject.
